(19) United States
(12) Patent Application Publication
(10) Pub. No.: US 2025/0180557 A1
(43) Pub. Date: Jun. 5, 2025

(54) ORAL MESENCHYMAL STEM CELLS AND USE THEREOF

(71) Applicant: RAMOT AT TEL-AVIV UNIVERSITY LTD., Tel Aviv (IL)

(72) Inventors: Evgeny WEINBERG, Tel Aviv (IL); Carlos NEMCOVSKY, Tel Aviv (IL); Daniel Zvi BAR, Tel Aviv (IL)

(21) Appl. No.: 19/049,691

(22) Filed: Feb. 10, 2025

Related U.S. Application Data
(63) Continuation of application No. PCT/IL2023/050861, filed on Aug. 15, 2023.
(60) Provisional application No. 63/398,014, filed on Aug. 15, 2022.

(51) Int. Cl. G01N 33/569; A61K 35/28; C12N 5/0775; G01N 33/68

(52) U.S. Cl. CPC G01N 33/56966; A61K 35/28; C12N 5/0668; G01N 33/6887; G01N 2333/47; G01N 2333/4703; G01N 2333/4704; G01N 2333/4706; G01N 2333/4712; G01N 2333/4716; G01N 2333/4727; G01N 2333/70578; G01N 2333/91011; G01N 2333/91045; G01N 2333/9125; G01N 2333/988

(57) ABSTRACT

A method for identification of a multipotent mesenchymal stem cell subpopulation, among a heterogenic population of gastrointestinal tract mucosa mesenchymal stromal cells, is provided. Further provided is a kit including a probe for identification or isolation of gastrointestinal tract mucosa mesenchymal stem cells, and a pharmaceutical composition including enriched or isolated mesenchymal stem cell from the gastrointestinal tract mucosa, for treating an individual suffering from a disorder or a disease.

### CROSS-REFERENCE TO RELATED-APPLICATIONS

This application is a ByPass Continuation of PCT Patent Application No. PCT/IL2023/050861 having International filing date of Aug. 15, 2023, which claims the benefit of priority of U.S. Provisional Patent Application No. 63/398,014, titled “ORAL MESENCHYMAL STEM CELLS AND USE THEREOF” filed 15 Aug. 2022, the contents of which are all incorporated herein by reference in their entirety.

### FIELD OF INVENTION

The present invention is in the field of stem cells.

### BACKGROUND

Mesenchymal stromal cells are a subpopulation of heterogeneous non-hematopoietic fibroblast-like cells, exhibiting varying multipotential and immunomodulatory capacities. More specifically, mesenchymal stromal cells are defined as bulk population that meet the following minimal criteria: capacity of plastic adherence, expression of specific biomarkers such as CD73 (NT5E), CD90 (THY1) and CD105 (ENG), lack of expression of hematopoietic and endothelial markers such as CD34, CD45 and HLA-DR, and capability of multipotent in vitro differentiation into adipocyte, chondrocyte and osteoblast lineages. Cells derived from the lamina propria of human oral mucosae have consistently shown high expression of mesenchymal stromal cells markers and various degrees of plasticity in vitro, and are, therefore, suitable for being called oral mucosa-derived mesenchymal stromal cells.

Owing to their multi-potency and plasticity, potent immunomodulatory/anti-inflammatory and immunosuppressive capacities, their ease of access by means of minimally invasive procedures that do not generate aesthetic problems, and efficient culturing methods, the ex vivo-expanded oral mucosa-derived mesenchymal stromal cells represent a resource of great interest for regenerative approaches.

Oral mucosa-derived mesenchymal stromal cells have been extensively investigated as regenerative therapeutics for the treatment of a wide range of pathological conditions and diseases, such as skin disorder, autoimmune and inflammatory diseases and nerve regeneration. Experimental therapeutic approaches were also conducted with these cells for periodontal regeneration, demonstrating a remarkable regenerative potential in experimental animal models in vivo. Oral mucosa-derived mesenchymal stromal cells were also employed for the treatment of peri-implantitis, maxillo-facial and calvarial bone defects, gingival defects and chemotherapy-related oral mucositis.

However, it is likely that the cultures of mesenchymal stromal cells contain heterogeneous mixtures of cells with distinct properties, including fibroblasts, myofibroblasts (fibroblasts with characteristics of smooth muscle cells) and a small proportion of the rarer progenitor/stem cell populations (e.g., mesenchymal stem cells, MSCs). There is an unmet need for identification of specific cell markers to distinguish rare cell sub-populations, such as mesenchymal stem cells, from the general population of mesenchymal stromal cells, from either masticatory or lining oral mucosa. There is further an unmet need for therapeutic use of enriched subpopulation of mesenchymal stem cells, such as oral mucosa derived mesenchymal stem cells.

### SUMMARY

According to a first aspect there is provided a method for identifying a multipotent mesenchymal stem cell subpopulation, among a population of cells, comprising the steps of: i. providing the population of cells; and ii. contacting and determining the population of cells with at least one probe, capable of identifying at least one marker, selected from a group consisting of: ubiquitin conjugating enzyme E2 S (UBE2S), assembly factor for spindle microtubules (ASPM), protein regulator of cytokinesis 1 (PRC1), ubiquitin conjugating enzyme E2 C (UBE2C), fatty acid binding protein 5 (FABP5), TNF receptor superfamily member 12A (TNFRSF12A), S100 calcium binding protein A16 (S100A16), ornithine decarboxylase 1 (ODC1), enolase 1 (ENO1), NME/NM23 nucleoside diphosphate kinase 1 (NME1), centrosomal protein 55 (CEP55), diaphanous related formin 3 (DIAPH3), Y-box binding protein 1 (YBX1), complement C1q binding protein (C1QBP), eukaryotic translation initiation factor 5A (EIF5A), DnaJ heat shock protein family (DNAJC9), 5′-nucleotidase ecto (NT5E), Rho GTPase activating protein 18 (ARHGAP18), UDP-N-acetylglucosamine pyrophosphorylase 1 (UAP1), actin related protein 2/3 complex subunit 1A (ARPC1A), lymphocyte antigen 6 family member K (LY6K), ezrin (EZR), Rho GTPase activating protein 22 (ARHGAP22), PDGFA associated protein 1 (PDAP1), epithelial membrane protein 3 (EMP3), caveolae associated protein 3 (CAVIN3), proteasome 26S subunit, ATPase 5 (PSMC5), chaperonin containing TCP1 subunit 3 (CCT3), COMM domain containing 4 (COMMD4), annexin A2 (ANXA2), proteasome 26S subunit, non-ATPase 9 (PSMD9), coordinator of PRMT5 and differentiation stimulator (COPRS), PALM2 and AKAP2 fusion (PALM2-AKAP2), cytoskeleton associated protein 5 (CKAP5), CDC42 effector protein 3 (CDC42EP3), AXL receptor tyrosine kinase (AXL), moesin (MSN), WD repeat domain 1 (WDR1), DNA methyltransferase 1 (DNMT1), high mobility group box 2 (HMGB2), nestin (NES), proliferating cell nuclear antigen (PCNA), proteasome 26S subunit, non-ATPase 2 (PSMD2), Transmembrane Protein 158 (TMEM158), Transmembrane 4 L Six Family Member 1 (TM4SF1), and any combination thereof, thereby, identifying a multipotent mesenchymal stem cell subpopulation, among a population of cells.

According to another aspect, there is provided a pharmaceutical composition comprising a mesenchymal stem cell subpopulation, and an acceptable carrier, wherein the mesenchymal stem cell subpopulation: (a) constitutes at least 50% of cells within the pharmaceutical composition; and (b) is characterized by expression of at least one marker, selected from a group consisting of: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, NT5E, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2 TMEM158, TM4SF1, and any combination thereof.

According to another aspect, there is provided a method for treating an individual suffering from a disorder or a disease, in need thereof, with the pharmaceutical composition disclosed herein.

In some embodiments, the population of cells comprises gastrointestinal tract mucosa mesenchymal stromal cells, comprising the multipotent mesenchymal stem cell, and at least one of: a fibroblast and a myofibroblast cell.

In some embodiments, the gastrointestinal tract is the oral cavity.

In some embodiments, the mucosa is the lamina propria.

In some embodiments, the lamina propria is from a masticatory oral mucosa.

In some embodiments, the population of cells is provided by separation of the connective tissue comprising the lamina propria, from the epithelium of the mucosal tissue.

In some embodiments, the lamina propria is from a masticatory oral mucosa.

In some embodiments, contacting is with a panel of probes, identifying a plurality of markers selected from a group consisting of: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2, TMEM158, TM4SF1, and any combination thereof.

In some embodiments, identifying comprises isolating.

In some embodiments, isolating is by cell sorting.

In some embodiments, the probe comprises one of: an antibody, an antibody fragment, a small molecule, and any combination thereof, conjugated to detectable tracer, comprising: a fluorophore, a chromophore, a magnetic bead, or any combination thereof.

In some embodiments, the method is for treating a disorder or a disease in a subject in need thereof.

In some embodiments, the disorder or disease in a subject in need thereof, is selected from: a skin disorder, an autoimmune or an inflammatory disease, nerve damage, an oral or craniofacial disorder, and a bone or a cartilage disorder.

In some embodiments at least one probe is a panel of probes, identifying a plurality of markers selected from a group consisting of: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2, TMEM158, and TM4SF1.

In some embodiments, the mesenchymal stem cell subpopulation comprises: a multipotent phenotype, an anti-inflammatory characteristic, an immunomodulatory characteristic, or any combination thereof.

In some embodiments, a multipotent phenotype is an ability to differentiate into at least an osteogenic lineage.

In some embodiments, the expression is, independently, above a predetermined threshold.

In some embodiments, the above a predetermined threshold expression, is at least 1.2-fold induction in the expression, compared to expression in a mesenchymal stromal non-stem cell, within the composition, comprising: a fibroblast, a myofibroblast, or any combination thereof.

In some embodiments, the pharmaceutical composition is characterized by above a predetermined threshold expression of a plurality of markers selected from a group consisting of: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2, TMEM158, and TM4SF1.

In some embodiments, the disorder or a disease is selected from: a skin disorder, an autoimmune or an inflammatory disease, a nerve damage, an oral or craniofacial disorder, and a bone or a cartilage disorder.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B include graphs demonstrating single-cell transcriptome profiling of mesenchymal stromal cells derived from masticatory and lining oral mucosa: (1A) UMAP plot showing distribution of all the cells according to their origin (L—of lining and M of masticatory oral mucosa origin); (1B) Cell cluster identification on the UMAP plot.

FIG. 2 includes a graph demonstrating UMAP plot showing the distribution of the cells constituting cluster #8, according to their origin (L—of lining and M of masticatory oral mucosa origin).

FIGS. 3A-3B includes graphs showing (3A) cell cluster annotation using Single R overlaid on the UMAP plot; (3B) 100% Stacked column diagram of the distribution of smooth muscle cells, fibroblasts, and mesenchymal stem cells (MSC) in the two tissue types (L—of lining and M of masticatory oral mucosa origin).

FIG. 4 includes violin plots showing the relative expression levels of typical mesenchymal stromal cell markers in each cluster.

FIG. 5 includes a dot plot presenting the summary of GO enrichment analysis for biological processes according to the different clusters: Top GO processes (according to p-value) for each cluster are shown. The size of the dots represents the gene ratios (count of cluster-specific markers/count of the pathway genes associated with the GO term), and the color of the dots represents the p-adjusted values (Fisher's exact test).

FIG. 6 includes a horizontal bar graph demonstrating the summary of GO enrichment analysis for biological processes in cluster #1. The size of the bars represents the number of the identified cluster-specific markers in the significant DE gene list associated with the GO term, and the color of the bars represents the p-adjusted values (Fisher's exact test).

FIG. 7 includes a table summarizing the number of cells per sample.

FIGS. 8A-8C include micrographs (8A), a table (8B), and photographs (8C) showing comparison of expression levels of various gene groups between cells derived from lamina propria of either masticatory (MM) or lining (LM) oral mucosa.

### DETAILED DESCRIPTION

The present invention, in some embodiments, provides a method for identification and/or selection of a multipotent mesenchymal stem cell subpopulation, such as among a heterogenic population of gastrointestinal tract mucosa mesenchymal stromal cells, comprising the mesenchymal stem cell, and at least one of: a fibroblast and a myofibroblast.

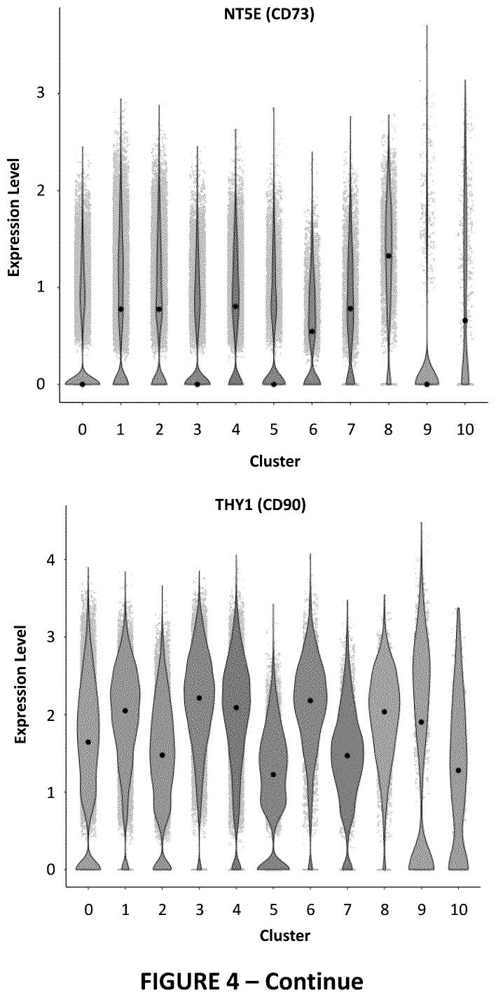

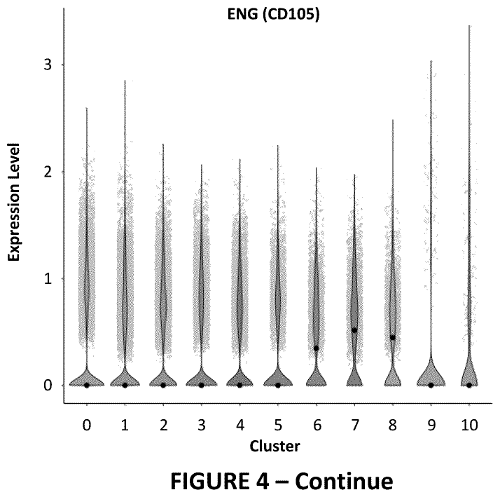

As demonstrated herein below, it was discovered that mesenchymal stomal cells, for example derived from the oral mucosa, are heterogenous, and comprise a subpopulation of mesenchymal stem cells, e.g., cells with multipotent capacity, as well as non-stem cells. In some embodiments, the non-stem cells among the mesenchymal stromal cells, comprise fibroblasts and/or myofibroblasts. In some embodiments, as demonstrated in FIG. 4, the inventors have found that the common markers used for identifying mesenchymal stem cells, comprising: CD44, CD73 (NT5E), CD90 (THY1), or CD105 (ENG), are not specific for mesenchymal stem cells, and cannot distinguish between mesenchymal stem cells and non-stem cells, among the mesenchymal stromal cells. In some embodiments, as described in table 1, the inventors further provide unique/specific markers, that can be used, for mesenchymal stem cells identification/isolation. In some embodiments, this method can be used for specific enhancement or isolation of mesenchymal stem cell, for mesenchymal stem cell therapy, in a subject in need thereof.

The present invention, in some embodiments, provides a method for identification of a multipotent mesenchymal stem cell subpopulation, among a population of cells.

In some embodiments, the method comprises the steps of:
i. providing a cell population derived from a mucosal tissue of the gastrointestinal tract; and,
ii. contacting the cell population with at least one probe, capable of identifying at least one marker, selected from: ubiquitin conjugating enzyme E2 S (UBE2S), assembly factor for spindle microtubules (ASPM), protein regulator of cytokinesis 1 (PRC1), ubiquitin conjugating enzyme E2 C (UBE2C), fatty acid binding protein 5 (FABP5), TNF receptor superfamily member 12A (TNFRSF12A), S100 calcium binding protein A16 (S100A16), ornithine decarboxylase 1 (ODC1), enolase 1 (ENO1), NME/NM23 nucleoside diphosphate kinase 1 (NME1), centrosomal protein 55 (CEP55), diaphanous related formin 3 (DIAPH3), Y-box binding protein 1 (YBX1), complement C1q binding protein (C1QBP), eukaryotic translation initiation factor 5A (EIF5A), DnaJ heat shock protein family (DNAJC9), Rho GTPase activating protein 18 (ARHGAP18), UDP-N-acetylglucosamine pyrophosphorylase 1 (UAP1), actin related protein 2/3 complex subunit 1A (ARPC1A), lymphocyte antigen 6 family member K (LY6K), ezrin (EZR), Rho GTPase activating protein 22 (ARHGAP22), PDGFA associated protein 1 (PDAP1), epithelial membrane protein 3 (EMP3), caveolae associated protein 3 (CAVIN3), proteasome 26S subunit, ATPase 5 (PSMC5), chaperonin containing TCP1 subunit 3 (CCT3), COMM domain containing 4 (COMMD4), annexin A2 (ANXA2), proteasome 26S subunit, non-ATPase 9 (PSMD9), coordinator of PRMT5 and differentiation stimulator (COPRS), PALM2 and AKAP2 fusion (PALM2-AKAP2), cytoskeleton associated protein 5 (CKAP5), CDC42 effector protein 3 (CDC42EP3), AXL receptor tyrosine kinase (AXL), moesin (MSN), WD repeat domain 1 (WDR1), DNA methyltransferase 1 (DNMT1), high mobility group box 2 (HMGB2), nestin (NES), proliferating cell nuclear antigen (PCNA), and proteasome 26S subunit, non-ATPase 2 (PSMD2), Transmembrane Protein 158 (TMEM158), Transmembrane 4 L Six Family Member 1 (TM4SF1), or any combination thereof, thereby, identifying a multipotent mesenchymal stem cell subpopulation, among a population of cells.

In some embodiments, the population of cells comprises a heterogenic population of gastrointestinal tract mucosa mesenchymal stromal cells. In some embodiments, the mesenchymal stromal cells comprise mesenchymal stem cell, and at least one of: a fibroblast and a myofibroblast.

### Definitions

Mesenchyme is an embryonic connective tissue that is derived from the mesoderm and that differentiates into hematopoietic and connective tissue. However, it should be noted that mesenchymal stem cells do not naturally differentiate into hematopoietic cells.

Stromal cells are connective tissue cells that form the supportive structure in which the functional cells of the tissue reside.

As used herein, the term “mesenchymal stomal cells” refers to a heterogenous population of cells, derived from a mesenchyme connective tissue, comprising: a mesenchymal stem cell, and at least one of: a fibroblast and a myofibroblast.

As used herein, the term “mesenchymal stem cell” (or MSC) refers to multipotent, stem cell capable of differentiating into at least one cell lineage. In some embodiments, the mesenchymal stem cell is capable to differentiate to varieties of mature cell types, comprising osteoblasts, chondrocytes, adipocytes, tenocytes, myotubes, neural cells, or hematopoietic-supporting stroma, either in vitro or in vivo. In some embodiments, mesenchymal stem cells are a subpopulation of mesenchymal stromal cells, exhibiting a multipotent capability. In some embodiments, mesenchymal stem cell is also referred to as multipotent mesenchymal stromal cell. In some embodiments, mesenchymal stem cells are primarily found in the stromal component of the bone marrow and many other tissues such as gastrointestinal tract, umbilical cord, and adipose. In some embodiments, mesenchymal stem cell phenotypically is fibroblast-like and/or plastic-adherent. In some embodiments, mesenchymal stem cell has the potency to self-renew and/or differentiate into distinct types of cell lineages. As used herein, the term “multipotent” refers to an ability of a cell to differentiate into more than one cell type.

In some embodiments, the mesenchymal stromal cells are derived from a gastrointestinal tract mucosa. As used herein, the term “mucosa”, or a mucous membrane, comprises a membrane that lines various cavities in the body of an organism (e.g., the digestive, respiratory, or reproductive tracts). In some embodiments, the mucosa comprises a layer of loose connective tissue, named lamina propria. In some embodiments, the mucosa comprises one or more layers of epithelial cells that secrete mucus, overlying the lamina propria. In some embodiments, the term “lamina propria” comprises a thin layer of loose (areolar) connective tissue. In some embodiments, the lamina propria lies beneath the epithelium, and together with the epithelium and basement membrane constitutes the mucosa.

In some embodiments, the gastrointestinal tract comprises the upper gastrointestinal tract, comprising oral cavity, salivary glands, esophagus, stomach, or small intestine (duodenum, jejunum, or ileum).

In some embodiments, the method comprises identification of at least one of the markers selected from: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, NT5E, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2, TMEM158, or TM4SF1.

Ubiquitin conjugating enzyme E2 S (UBE2S) comprises an enzyme that is a member of the E2 ubiquitin-conjugating enzyme family. UBE2S in humans (UniProtKB #Q16763) is encoded by the UBE2C gene (gene ID #27338). In some embodiments, UBE2S enzyme is transferase that accepts ubiquitin from the E1 complex and catalyzes its covalent attachment to other proteins. In some embodiments, UBE2S comprises intracellular location.

In some embodiments cellular location of EBE2S comprises the nucleoplasm, the plasma membrane, or the cytosol. In some embodiments, there are at least 2 potential isoforms mapped to UBE2S (e.g., UniProtKB #K7EM75 or K7EPJ1). Assembly factor for spindle microtubules (ASPM) is also known as abnormal spindle-like microcephaly-associated protein, or abnormal spindle protein homolog, or Asp homolog. ASPM protein in humans (UniProtKB #Q8IZT6) is encoded by the ASPM gene (gene ID #259266). In some embodiments, ASPM protein is involved in mitotic spindle regulation and coordination of mitotic processes. In some embodiments, ASPM comprises intracellular location. In some embodiments, ASPM cellular location comprises: plasma membrane or cytosol. In some embodiments, multiple transcript variants encoding different isoforms have been found for ASPM gene. In some embodiments, ASPM comprises at least 2 alternative splicing isoforms (e.g., UniProtKB #Q8IZT6-1 or Q8IZT6-2).

Protein regulator of cytokinesis 1 (PRC1) is a protein that in humans (UniProtKB #043663) is encoded by the PRC1 gene (gene ID #9055). In some embodiments, PRC1 is involved in cytokinesis. In some embodiments, PRC1 protein is present at high levels during the S and G2/M phases of mitosis and its levels drop dramatically when the cell exits mitosis and enters the G1 phase. In some embodiments, PRC1 is located in the nucleus during interphase, becomes associated with mitotic spindles during mitosis, and localizes to the cell midbody during cytokinesis. In some embodiments, PRC1 protein comprises intracellular location. In some embodiments, PRC1 cellular location comprises nucleoplasm, plasma membrane, microtubules, cytokinetic bridge, or midbody. In some embodiments, PRC1 comprises at least 4 alternative splice isoforms (UniProtKB #043663-1, 043663-2, 043663-3, or 043663-4). In some embodiments, PRC1 comprises at least 6 additional potential isoforms (e.g., UniProtKB #HOYNE4, HOYM42, H0YL53, H0YLA0, H3BSJ8, or G3V3N7).

Ubiquitin conjugating enzyme E2 C (UBE2C) comprises an enzyme that is a member of the E2 ubiquitin-conjugating enzyme family. UBE2C in humans (UniProtKB #000762) is encoded by the UBE2C gene (gene ID #11065). In some embodiments, UBE2C enzyme is transferase that accepts ubiquitin from the E1 complex and catalyzes its covalent attachment to other proteins. In some embodiments, UBE2C comprises intracellular location. In some embodiments, the cellular location of UBE2C comprises plasma membrane or cytosol. In some embodiments, multiple alternatively spliced transcript variants have been found for UBE2C. In some embodiments, UBE2C comprises at least 4 isoforms produced by alternative splicing (e.g., UniProtKB #000762-1, 000762-2, 000762-3, or 000762-4). In some embodiments, there are at least 3 potential isoforms mapped to UBE2C (e.g., UniProtKB #K4DI81, A0A0A0MSE8, or A0A087WVK1).

Fatty acid binding protein 5 (FABP5), also known as fatty acid-binding protein, epidermal comprises a protein that binds long-chain fatty acid or other hydrophobic ligands. FABP5 in humans (UniProtKB #Q01469) is encoded by the FABP5 gene (gene ID #2171).

In some embodiments, FABP5 comprises intracellular location. In some embodiments cellular location of FABP5 comprises the plasma membrane or the cytosol. In some embodiments, there are at least 2 potential isoforms mapped to FABP5 (e.g., UniProtKB #Q01469 or I6L8B7).

Tumor necrosis factor TNF receptor superfamily member 12A (TNFRSF12A), also known as TWEAK receptor or CD266 antigen, is a receptor that in humans (UniProtKB #Q9NP84) is encoded by the TNFRSF12A gene (gene ID #51330). TNFRSF12A is a receptor for TNFSF12/TWEAK. In some embodiments, TNFRSF12A function comprises weak induction of apoptosis in some cell types, induction of angiogenesis and/or proliferation of endothelial cells, or modulation of cellular adhesion to matrix proteins. In some embodiments, TNFRSF12A comprises intracellular expression (e.g., cytosol). In some embodiments, TNFRSF12A comprises location on the cell surface membrane. In some embodiments, TNFRSF12A comprises at least 2 isoforms produced by alternative splicing (e.g., UniProtKB #Q9NP84-1 or Q9NP84-2). In some embodiments, there are at least 4 potential isoforms mapped to TNFRSF12A (e.g., UniProtKB #I3L539, I3L3P4, I3L1J9, or I3LOS4).

S100 calcium binding protein A16 (S100A16), is a protein that in humans (UniProtKB #Q96FQ6) is encoded by the S100A16 gene (gene ID #140576). In some embodiments, S100A16 comprises intracellular location. In some embodiments, cellular location comprises plasma membrane or cytosol.

Ornithine decarboxylase 1 (ODC1) is an enzyme that in humans (UniProtKB #P11926) is encoded by the ODC1 gene (gene ID #4953). In some embodiments, ODC1 protein is a rate-limiting enzyme that catalyzes the first and rate-limiting step of polyamine biosynthesis and converts ornithine into putrescine (the precursor for polyamine, spermidine and spermine). In some embodiments, polyamine is essential for cell proliferation and is implicated in cellular processes, ranging from DNA replication to apoptosis. In some embodiments, ODC1 protein comprises intracellular location. In some embodiments, ODC1 cellular location comprises: plasma membrane, or cytosol. In some embodiments, multiple alternatively spliced transcript variants encoding distinct isoforms of ODC1 have been identified. In some embodiments, ODC1 comprises at least two alternative splice isoforms (e.g., UniProtKB #P11926, or C9JG30).

Enolase 1 (ENO1), also known as alpha-enolase, is an enzyme that in humans (UniProtKB #P06733) is encoded by the ENO1 gene (gene ID #2023). In some embodiments, ENO1 is a glycolytic enzyme that is one of the isozymes of enolase. In some embodiments, isoenzyme is a homodimer composed of 2 alpha, 2 gamma, or 2 beta subunits, and functions as a glycolytic enzyme. In some embodiments, ENO1 comprises intracellular location. In some embodiments, cellular location comprises plasma membrane or cytosol. In some embodiments, alternative splicing of this gene results in a shorter isoform that function as a tumor suppressor. In some embodiments, several pseudogenes have been identified, including one on the long arm of chromosome 1.

NME/NM23 Nucleoside Diphosphate Kinase 1 (NME1), also named nucleoside diphosphate kinase A, is an enzyme that in humans (UniProtKB #P15531) is encoded by the NME1 gene (gene ID #4830). In some embodiments, nucleoside diphosphate kinase (NDK) exists as a hexamer composed of ‘A’ (encoded by this NME1 gene) and ‘B’ (encoded by NME2) isoforms. In some embodiments, co-transcription of NME1 gene and NME2 gene generates naturally occurring transcripts (NME1-NME2), which encodes a fusion protein consisting of sequence sharing identity with each individual gene product. In some embodiments, NME1 protein has a major role in the synthesis of nucleoside triphosphates other than ATP. In some embodiments, NME1 comprises intracellular location. In some embodiments, NME1 cellular location comprises: nucleoplasm, plasma membrane, or cytosol. In some embodiments NME1 comprises at least 4 alternative splicing isoforms (e.g., UniProtKB #P15531, E7ERL0, C9K028, or E5RHP0).

Centrosomal protein of 55 kDa (CEP55) in humans (UniProtKB #Q53EZ4) is encoded by the CEP55 gene (gene ID #55165). In some embodiments, CEP55 is a mitotic phosphoprotein that plays a key role in cytokinesis, the final stage of cell division. In some embodiments, CEP55 comprises intracellular location. In some embodiments, CEP55 cellular location comprises: plasma membrane, midbody, or centriolar satellite. In some embodiments, CEP55 comprises at least 2 alternative splicing isoforms (e.g., UniProtKB #Q53EZ4-1 or Q53EZ4-2).

Diaphanous related formin 3 (DIAPH3) in humans (UniProtKB #Q9NSV4) is encoded by the DIAPH3 gene (gene ID #81624). In some embodiments, DIAPH3 is a member of the diaphanous subfamily of the formin family. In some embodiments, DIAPH3 is involved in actin remodeling and regulation of cell movement or adhesion. In some embodiments, multiple transcript variants encoding different isoforms have been found for DIAPH3. In some embodiments, DIAPH3 comprises intracellular location. In some embodiments, DIAPH3 cellular location comprises: plasma membrane microtubules.

Y-box binding protein 1 (YBX1), also known as Y-box transcription factor or nuclease-sensitive element-binding protein 1, is a protein that in humans (UniProtKB #P67809) is encoded by the YBX1 gene (gene ID #4904). In some embodiments, YBX1 is a DNA- and RNA-binding protein involved in various processes, such as translational repression, RNA stabilization, mRNA splicing, DNA repair and transcription regulation. In some embodiments, YBX1 comprises intracellular location. In some embodiments, YBX1 cellular location comprises endoplasmic reticulum, vesicles, plasma membrane, or cytosol. In some embodiments, multiple transcript variants encoding different isoforms have been found for YBX1.

Complement C1q binding protein (C1QBP), also known as complement component 1 Q subcomponent-binding protein mitochondrial, is a protein that in humans (UniProtKB #Q07021) is encoded by the C1QBP gene (gene ID #708). In some embodiments, C1QBP is known to associate with C1r and/or C1s in order to yield the first component of the serum complement system. In some embodiments, C1QBP comprises intracellular location. In some embodiments, C1QBP is secreted to blood (different isoforms). In some embodiments, cellular location comprises the plasma membrane or the mitochondria. In some embodiments, multiple transcript variants encoding different isoforms have been found for C1QBP (e.g., UniProtKB #I3L3B0, I3L3Q7 or A0A0G2JLC0).

Eukaryotic translation initiation factor 5A (EIF5A), or eukaryotic translation initiation factor 5A-1, is a protein that in humans (UniProtKB #P63241) is encoded by the EIF5A gene (gene ID #1984). In some embodiments, EIF5A is unique by containing the unusual amino acid hypusine. In some embodiments, EIF5A is a mRNA-binding protein involved in translation elongation. In some embodiments, EI5FA comprises intracellular location. In some embodiments, cellular location comprises the nucleoplasm, the plasma membrane, or the cytosol. In some embodiments, there at least two different isoforms for EIF5A (e.g., UniProtKB #I3L504 or I3L397).

DnaJ heat shock protein family (DNAJC9), or DNAJ (Hsp40) homolog, subfamily C, member 9, is a protein that in humans (UniProtKB #Q8WXX5) is encoded by the DNAJC9 gene (gene ID #23234). In some embodiments, DNAJC9 is part of the chaperone complex. In some embodiments, DNAJC9 comprises intracellular location. In some embodiments, DNAJC9 comprises an extracellular location. In some embodiments, the cellular location of DNAJC9 comprises the nucleoplasm, the cytosol or the plasma membrane.

Rho GTPase activating protein 18 (ARHGAP18) is a protein that in humans (UniProtKB #Q8N392) is encoded by the ARHGAP18 gene (gene ID #93663). In some embodiments, the gene is also known as MacGAP and bA307014.2. In some embodiments, ARHGAP18 belongs to a family of Rho GTPase-activating proteins that modulate cell signaling. In some embodiments, ARHGAP18 comprises intracellular location. In some embodiments, cellular location comprises the nuclear speckles, the plasma membrane, or the cytosol. In some embodiments, ARHGAP18 has at least 2 isoforms produced by alternative splicing (e.g. UniProtKB #Q8N392-1 or Q8N392-2).

UDP-N-acetylglucosamine pyrophosphorylase 1 (UAP1), also named antigen X (or AGX), is an enzyme that in humans (UniProtKB #Q16222) is encoded by the UAP1 gene (gene ID #6675). UAP1 enzyme function comprises: convertion UTP and GlcNAc-1-P into UDP-GlcNAc, or UTP and GalNAc-1-P into UDP-GalNAc. In some embodiments, isoform AGX1 has higher activity towards GalNAc-1-P, while isoform AGX2 has more activity towards GlcNAc-1-P. In some embodiments, UAP1 comprises intracellular expression. In some embodiments, UAP1 cellular location comprises: nucleoplasm, plasma membrane, or cytosol. In some embodiments, UAP1 comprises at least 3 alternative splice isoforms (UniProtKB #Q16222-1, Q16222-2, or Q16222-3).

Actin related protein 2/3 complex subunit 1A (ARPC1A), also named SOP2L, is a protein that in humans (UniProtKB #Q92747) is encoded by the ARPC1A gene (gene ID #10552). In some embodiments, ARPC1A is one of seven subunits of the human Arp2/3 protein complex. In some embodiments, this subunit is a member of the SOP2 family of proteins. In some embodiments, ARPC1A is an actin-binding protein. In some embodiments, ARPC1A comprises intracellular location. In some embodiments, the cellular location of ARPC1A comprises the nucleus, the cell junction, or the cytosol. In some embodiments, cellular location comprises the plasma membrane. In some embodiments, ARPC1A has at least 2 isoforms produced by alternative splicing (Q92747-1 or Q92747-2).

Lymphocyte antigen 6 family member K (LY6K) is a protein that in humans (UniProtKB #Q17RY6) is encoded by the LY6K gene (gene ID #54742). In some embodiments, LY6K plays a role in cell growth. In some embodiments, LY6K is expressed on the cell surface of sperm and involved in binding activity of sperm to zona pellucida. In some embodiments, LY64 location comprises the cellular membrane. In some embodiments cellular location comprises the cytoplasm. In some embodiments, LY6K is secreted (different isoforms). In some embodiments, LY6K has at least 2 isoforms produced by alternative splicing (e.g., Q17RY6-1 and Q17RY6-2).

Ezrin (EZR) is also known as cytovillin or villin-2, and in humans (UniProtKB #P15311) is encoded by the EZR gene (gene ID #7430). Ezrin protein is a member of the ERM protein family, comprising ezrin, moesin and radixin. In some embodiments, ezrin protein is a cytoplasmic peripheral membrane protein (i.e., a protein that adhere only temporarily to the biological transmembrane). In some embodiments, EZR functions as a protein-tyrosine kinase substrate in microvilli. In some embodiments, ezrin has key roles in cell surface structure, adhesion, migration, or organization. In some embodiments, ezrin protein comprises intracellular location. In some embodiments, ezrin cellular location comprises the plasma membrane. In some embodiments, alternatively spliced variants have been described for EZR.

Rho GTPase activating protein 22 (ARHGAP22) is a protein that in humans (UniProtKB #Q7Z5H3) is encoded by the ARHGAP22 gene (gene ID #58504). In some embodiments, ARHGAP22 is a member of the GTPase activating protein family. In some embodiments, ARHGAP22 protein comprises intracellular location. In some embodiments, ARHGAP22 cellular location comprises the plasma membrane, the nucleoplasm, or the cytosol. In some embodiments, ARHGAP22 comprises at least 5 isoforms produced by alternative splicing (e.g., UniProtKB #Q7Z5H3-1, Q7Z5H3-2, Q7Z5H3-3, Q7Z5H3-4, or Q7Z5H3-5).

PDGFA associated protein 1 (PDAP1), also named 28 kDa heat- and acid-stable phosphoprotein is a protein that in humans (UniProtKB #Q13442) is encoded by the PDAP1 gene (gene ID #11333). In some embodiments, PDAP1 is a phosphoprotein that may upregulate the PDGFA-stimulated growth of fibroblasts. In some embodiments, PDAP1 comprises intracellular location. In some embodiments, PDAP1 comprises extracellular location. In some embodiments, the cellular location of PDAP1 comprises the plasma membrane or the cytosol. In some embodiments, there is at least one isoform produced by alternative splicing (e.g., UniProtKB #F8WBW6).

Epithelial membrane protein 3 (EMP3) is a protein that in humans (UniProtKB #P54852) is encoded by the EMP3 gene (gene ID #2014). Is some embodiments, EMP3 protein contains four transmembrane domains and two N-linked glycosylation sites. In some embodiments, EMP3 comprises intracellular location. In some embodiments, EMP3 comprises membrane location (different isoforms). In some embodiments, the cellular location of EMP3 comprises the vesicle or the plasma membrane. In some embodiments, alternative splicing of EMP3 results in multiple transcript variants. In some embodiments, EMP3 has at least 5 potential isoforms (e.g., UniProtKB #MOR1L9, MOR122, MOQXN1, MOQXSO, or MOQZ66).

Caveolae associated protein 3 (CAVIN3) is a protein that in humans (UniProtKB #Q969G5) is encoded by the CAVIN3 gene (gene ID #112464). In some embodiments, CAVIN3 is a binding protein of the protein kinase C, delta (PRKCD). In some embodiments, CAVIN3 regulates the traffic and/or budding of caveolae. In some embodiments, CAVIN3 comprises intracellular location. In some embodiments, cellular location comprises the plasma membrane or the cytoplasm. In some embodiments, there is at least 1 potential isoform mapped to CAVIN3 (e.g., UniProtKB #E9PIE3).

Proteasome 26S subunit, ATPase 5 (PSMC5), also known as 26S protease regulatory subunit 8, or as 26S proteasome AAA-ATPase subunit Rpt6, is an enzyme that in humans (UniProtKB #P62195) is encoded by the PSMC5 gene (gene ID #5705). In some embodiments, PSMC5 is a component of the 26S proteasome, a multiprotein complex involved in the ATP-dependent degradation of ubiquitinated proteins. In some embodiments, PSMC5 comprises an intracellular location. In some embodiments cellular location of PSMC5 comprises the plasma membrane, the nucleus, or the cytosol. In some embodiments, PSMC5 has at least 2 isoforms, produced by alternative splicing (e.g., UniProtKB #P62195-1 or P62195-2).

Chaperonin containing TCP1 subunit 3 (CCT3), also known as T-complex protein 1 subunit gamma, is a protein that in humans (UniProtKB #P49368) is encoded by the CCT3 gene (gene ID #7203). In some embodiments, CCT3 is a molecular chaperone that is a member of the chaperonin containing TCP1 complex (CCT), also known as the TCP1 ring complex (TRiC). In some embodiments, CCT3 comprises intracellular location. In some embodiments, CCT3 cellular location comprises the plasma membrane or the cytosol. In some embodiments, CCT3 has at least 2 isoforms produced by alternative splicing (e.g., UniProtKB #P49368-1 or P49368-2).

COMM Domain containing 4 (COMMD4) in humans (UniProtKB #Q9HOA8) is encoded by the COMMD4 gene (gene ID #54939). In some embodiments, COMMD4 may modulate activity of cullin-RING E3 ubiquitin ligase (CRL) complexes. In some embodiments, COMMD4 down-regulates activation of NF-kappa-B. In some embodiments, COMMD4 protein comprises intracellular expression. In some embodiments, COMMD4 cellular location comprises: vesicle, plasma membrane, or cytosol. In some embodiments, COMMD4 comprises at least 3 alternative splice isoforms (e.g., UniProtKB #Q9HOA8-1, Q9HOA8-2, or Q9HOA8-3).

Annexin A2 (ANXA2), also known as annexin II, is a protein that in humans (UniProtKB #P07355) is encoded by the ANX42 gene. In some embodiments, ANXA2 is a member of the annexin family. In some embodiments, ANXA2 is a calcium-dependent phospholipid-binding protein family that plays a role in the regulation of cellular growth and/or in signal transduction pathways. In some embodiments, ANXA2 is a calcium-regulated membrane-binding protein. In some embodiments, ANXA2 comprises intracellular location. In some embodiments ANXA2 is secreted to blood (different isoforms). In some embodiments, cellular location comprises the plasma membrane or the cytosol. In some embodiments, ANXA2 is located in the melanosome. In some embodiments, multiple alternatively spliced transcript variants encoding different isoforms have been found for ANXA2. In some embodiments, there are at least 2 isoforms produced by alternative splicing (e.g., UniProtKB #P07355-1 or P07355-2).

Proteasome 26S subunit, non-ATPase 9 (PSMD9), also known as 26S proteasome non-ATPase regulatory subunit 9, is an enzyme that in humans (UniProtKB #000233) is encoded by the PSMD9 gene (gene ID #5715). In some embodiments, PSMD9 acts as a chaperone during the assembly of the 26S proteasome. In some embodiments, PSMD9 comprises intracellular location. In some embodiments, the cellular location of PSMD9 comprises the plasma membrane, the nucleus, or the cytosol. In some embodiments, PSMD9 has at least 3 isoforms produced by alternative splicing (e.g., UniProtKB #000233-1, 000233-2 or 000233-3).

Coordinator of PRMT5 and differentiation stimulator (COPRS), also named coordinator of PRMT5, differentiation stimulator, is a protein that in humans (UniProtKB #Q9NQ92) is encoded by the COPRS gene (gene ID #55352). In some embodiments, COPRS is a histone-binding protein. In some embodiments, COPRS comprises intracellular location.

In some embodiments, COPRS cellular location comprises the nucleoplasm, the plasma membrane, or the cytosol. In some embodiments, there are at least 4 potential isoforms for COPRS (e.g., UniProtKB #J3QRX4, J3QRX6, J3QRX9, or H9KV77).

PALM2 and AKAP2 fusion (PALM2-AKAP2) is a protein that in humans (UniProtKB #Q8IXS6 and Q9Y2D5) is encoded by PALM2AKAP2 gene (gene ID #445815). In some embodiments, PALM2AKAP2 gene encodes three distinct protein isoforms, the PALM2 isoform, the AKAP2 isoform and the PALM2-AKAP2 isoform. In some embodiments, PALM2AKAP2 gene belongs to the paralemmin downstream gene (PDG) family defined in PMID:22855693. In some embodiments, PALM2-AKAP2 protein comprises intracellular location. In some embodiments, PALM2-AKAP2 cellular location comprises: nucleoplasm, nuclear bodies, or plasma membrane. In some embodiments, paralemmin-2, or PALM2, comprises at least 2 alternative splice isoforms (e.g., UniProtKB #Q8IXS6-1 or Q8IXS6-2). In some embodiments, a-kinase anchor protein 2, or AKAP2, comprises at least 5 isoforms (e.g., UniProtKB #Q9Y2D5-3, Q9Y2D5-4, Q9Y2D5-5, Q9Y2D5-6, or Q9Y2D5-7).

Cytoskeleton-associated protein 5 (CKAP5) is a microtubule-associated protein that in humans (UniProtKB #Q14008) is encoded by the CKAP5 gene (gene ID #: 9793). In some embodiments, CKAP5 protein function comprises two distinct roles in spindle formation; it protects kinetochore microtubules from depolymerization or plays an essential role in centrosomal microtubule assembly. In some embodiments, CKAP5 protein comprises intracellular expression. In some embodiments, CKAP5 intracellular location comprises: nucleoli, nucleoli rim, plasma membrane, or centrosome. In some embodiments, CKAP5 comprises at least 3 alternative splicing isoforms (e.g., UniProtKB #Q14008-1, Q14008-2, or Q14008-3).

Cdc42 effector protein 3 (CDC42EP3) in humans (UniProtKB #Q9UKI2) is encoded by the CDC42EP3 gene (gene ID #10602). DCD42EP3 protein encoded by this gene is a member of the Borg family of CDC42 effector proteins. In some embodiments, CDC42 comprises a small Rho GTPase that regulates the formation of F-actin-containing structures. In some embodiments, the DCD42EP3 protein comprises intracellular expression. In some embodiments, DCD42EP3 protein cellular location comprises: plasma membrane or actin filaments. In some embodiments, alternative splicing results in multiple transcript variants of CDC42EP3. In some embodiments, CDC42EP3 comprises at least 4 alternative splicing isoforms (e.g., UniProtKB #Q9UKI2, C9JEZ4, C9J7F7 or C9JKW5).

AXL receptor tyrosine kinase, also known as tyrosine-protein kinase receptor UFO. AXL is a cell surface receptor tyrosine kinase that in humans (UniProtKB #P30530) is encoded by the AXL gene (gene ID #558). In some embodiments, AXL receptor tyrosine kinase transduces signals from the extracellular matrix into the cytoplasm by binding the growth factor GAS6, and thus regulates many physiological processes, including cell survival, cell proliferation, migration, and differentiation. In some embodiments, AXL receptor comprises location on the cell surface membrane. In some embodiments, AXL cellular location comprises: vesicles, or actin filaments. In some embodiments, alternative splicing results in multiple transcript variants of AXL gene. In some embodiments, AXL comprises at least 2 alternative splicing isoforms: (e.g., UniProtKB #P30530-1 or P30530-2).

Moesin (MSN), also named membrane-organizing extension spike protein and in humans (UniProtKB #P26038) is encoded by the MSN gene (gene ID #4478). In some embodiments, moesin protein is a member of the ERM protein family. In some embodiments, ERM proteins function as cross-linkers between plasma membranes and actin-based cytoskeletons. In some embodiments, moesin is localized to filopodia and other membranous protrusions that are important for cell-cell recognition, signaling, or cell movement. In some embodiments, moesin protein comprises intracellular location. In some embodiments, moesin cellular location comprises plasma membrane. In some embodiments, MSN comprises at least two alternative spliced isoforms (e.g., UniProtKB #P26038 or V9GZ54).

WD repeat-containing protein 1 (WDR1) is a protein that in humans (UniProtKB #075083) is encoded by the WDR1 gene (gene ID #9948). In some embodiments, WDR1 protein is an actin-binding protein which promotes induction of disassembly of actin filaments. In some embodiments, WDR1 comprises intracellular location. In some embodiments, cellular location comprises the plasma membrane or cell junction. In some embodiments, at least two transcript variants encoding different isoforms of WDR1 have been found (e.g., UniProtKB #075083-1 or 075083-3). In some embodiments, there is at least one potential isoform mapped to WDR1 (e.g., UniProtKB #D6RD66).

DNA Methyltransferase 1 (DNMT1) is also known as DNA (cytosine-5)-methyltransferase 1, and in humans (UniProtKB #P26358), it is encoded by the DNMT1 gene (gene ID #1786). DNMT1 is an enzyme that catalyzes the transfer of methyl groups to specific CpG structures in DNA, a process called DNA methylation. In some embodiments, DNMT1 protein comprises intracellular location. In some embodiments, DNMT1 cellular location comprises the nucleoplasm. In some embodiments, alternative splicing results in multiple transcript variants of DNMT1. In some embodiments there are at least 19 potential isoforms mapped to DNMT1.

High mobility group box 2 (HMGB2), also named high-mobility group protein B2 and in humans (UniProtKB #P26583) is encoded by the HMGB2 gene (gene ID #3148). This gene encodes a member of the non-histone chromosomal high mobility group protein family. In some embodiments, HMGB2 is a chromatin-associated multifunctional protein. In some embodiments, HMGB2 protein comprises intracellular location, comprising: nucleoplasm, or nucleoli. In some embodiments, HMGB2 protein comprises extracellular location, comprising isoform secreted to blood. In some embodiments, HMGB2 comprises at least two alternatively spliced isoforms (e.g., UniProtKB #P26583 or D6R9A6).

Nestin (NES) is a protein that in humans (UniProtKB #P48681) is encoded by the NES gene (gene ID #10763). In some embodiments, NES is a member of the intermediate filament protein family. In some embodiments, NES comprises intracellular location. In some embodiments, cellular location comprises the cytoplasm or the intermediate filament.

Proliferating cell nuclear antigen (PCNA) in humans (UniProtKB #P12004) is encoded by the PCNA gene (gene ID #5111). In some embodiments, PCNA protein is a DNA clamp that acts as a cofactor for DNA polymerase 6 (delta) in eukaryotic cells and is essential for replication. In some embodiments, PCNA has a key role in DNA synthesis or DNA repair. In some embodiments, PCNA protein comprises intracellular location. In some embodiments, PCNA cellular location comprises nucleoplasm. In some embodiments, two transcript variants encoding the same protein have been found for PCNA gene.

Proteasome 26S subunit ubiquitin receptor, non-ATPase 2 (PSMD2), also known as 26S proteasome regulatory subunit Rpn1, is an enzyme that in humans (UniProtKB #Q13200) is encoded by the PSMD2 gene (gene ID #5708). PSMD2 comprises multiprotein complex involved in the ATP-dependent degradation of ubiquitinated proteins. In some embodiments, the 26S proteasome has a highly ordered structure composed of 2 complexes, a 20S core and a 19S regulator. In some embodiments, PSMD2 comprises intracellular location. In some embodiments, PSMD2 comprises membranal expression. In some embodiments, PSMD2 is secreted or comprises extracellular location, such as extracellular exosome. In some embodiments, cellular localization comprises cytosol, proteasome storage granule, nucleus, nucleoplasm, ficolin-1-rich granule lumen, proteasome accessory complex, proteasome complex, proteasome regulatory particle, base subcomplex, or secretory granule lumen. In some embodiments, alternative splicing results in multiple transcript variants of the PSMD2 gene. In some embodiments, PSMD2 comprises at least 5 alternative splice isoforms (e.g., UniProtKB #Q13200, H7C1H2, C9JPC0, F8WBS8, or H7C2Q3).

Transmembrane protein 158 (TMEM) is a protein that in humans is encoded by the TMEM158 (UniProtKB #Q8WZ71).

Transmembrane 4 L6 family member 1 (TM4SF1) is a protein that in humans is encoded by the TM4SF1 gene (UniProtKB #P30408).

In some embodiments, the method comprises identification of an analog of least one of the markers selected from: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, NT5E, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2, TMEM158, TM4SF1, or any combination thereof. As used herein, the term “analog” refers to a protein or a nucleic acid having at least 80%, 90%, 95%, 99% identity or homology, or any value and range therebetween, to one of the abovementioned markers. Each possibility represents a separate embodiment of the invention.

In some embodiments, step (ii) of the method, i.e. contacting the cell population with at least one probe capable of identifying at least one marker, selected from: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2, TMEM158, or TM4SF1, is followed by a step of determining expression, or expression profile, of the marker. In some embodiments, identification of a mesenchymal stem cell is performed by expression of at least one of the abovementioned markers. In some embodiments, identification of a mesenchymal stem cell is performed by increased expression of at least one of the abovementioned markers, above a predetermined threshold.

The term “expression” as used herein refers to the biosynthesis of a gene product, including the transcription and/or translation of the gene product. Thus, expression of a nucleic acid molecule may refer to transcription of the nucleic acid fragment (e.g., transcription resulting in mRNA or other functional RNA) and/or translation of RNA into a precursor or mature protein (polypeptide). In some embodiments, expression comprises expression of differently alternatively spliced isoforms of the abovementioned markers.

As used in reference to the methods of the invention, “increased expression” refers to increase in expression of a marker, or a set of markers, above a predetermined threshold.

In some embodiments, a predetermined threshold comprises comparison to expression in a control cell. In some embodiments, the term “a control cell” refers to a mesenchymal stromal cell, non-stem cell, originated from same example of cell population as the examined mesenchymal stem cell. In some embodiments, the term “a control cell” refers to any other cell from identical species as the examined mesenchymal stem cell (e.g., human), either from the gastrointestinal tract tissue, or other reference tissue. For a non-limiting example, a specific value of increase may be a result of increase of all the markers of the set.

Alternatively, a specific value of increase may be a result of increase of only a few markers of the set, or a result of increase of a single marker of the set. In some embodiments, the increase refers to at least 10% increase, 20% increase, 30% increase, 40% increase, 50% increase, 60% increase, 70% increase, 80% increase, 90% increase, 100% increase in expression level, compared to a predetermined threshold. Each possibility represents a separate embodiment of the invention.

A variety of known techniques may be suitable for determining an expression profile. Such techniques include methods based on hybridization analysis of polynucleotides and on sequencing of polynucleotides, and proteomics-based methods. In some embodiments, the determining step is performed by nucleic acid hybridization, nucleic acid amplification, or an immunological method. In some embodiments, the determining step is performed in-situ. In some embodiments, fluorescence labeling or staining are applied. In some embodiments, an imaging step is further applied.

In some embodiments, the expression profile is obtained by measuring protein levels of the markers. In some embodiments, the expression, and the level of expression, of proteins or polypeptides of interest can be detected through immunohistochemical staining of tissue slices or sections. Additionally, proteins/polypeptides of interest may be detected by Western blotting, ELISA, Radioimmunoassay (RIA), or flow cytometry (FACS) assays employing protein-specific antibodies.

Alternatively, protein levels can be determined by constructing an antibody microarray in which binding sites comprise immobilized, preferably monoclonal, antibodies specific to a plurality of proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes).

In one embodiment, monoclonal antibodies are raised against synthetic peptide fragments designed based on genomic sequence of the cell. With such an antibody array, proteins from the cell are contacted to the array, and their binding is assayed with assays known in the art.

In some embodiments, the determining step comprises the step of obtaining nucleic acid molecules from the biological sample. In some embodiments, the nucleic acids molecules are selected from mRNA molecules, DNA molecules and cDNA molecules. In some embodiments, the cDNA molecules are obtained by reverse transcribing the mRNA molecules. In some embodiments, the expression profile is determined by measuring mRNA levels of the marker or set of markers. Methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995).

Numerous methods are known in the art for measuring expression levels of a one or more genes such as by amplification of nucleic acids (e.g., PCR, isothermal methods, rolling circle methods, etc.) or by quantitative in situ hybridization. The design of primers for amplification of specific genes is well known in the art, and such primers can be found or designed on various websites such as bioinfo.ut.ee/primer3-0.4.0/ or pga.mgh.harvard.edu/primerbank/ for example.

The skilled artisan will understand that these methods may be used alone or combined. Non-limiting exemplary method are described herein.

RT-qPCR: A common technology used for measuring RNA abundance is RT-qPCR where reverse transcription (RT) is followed by real-time quantitative PCR (qPCR). Reverse transcription first generates a DNA template from the RNA. This single-stranded template is called cDNA. The cDNA template is then amplified in the quantitative step, during which the fluorescence emitted by labeled hybridization probes or intercalating dyes changes as the DNA amplification process progresses. Quantitative PCR produces a measurement of an increase or decrease in copies of the original RNA and has been used to attempt to define changes of gene expression in cancer tissue as compared to comparable healthy tissues.

RNA-Seq: RNA-Seq uses recently developed deep-sequencing technologies. In general, a population of RNA (total or fractionated, such as poly(A)+) is converted to a library of cDNA fragments with adaptors attached to one or both ends. Each molecule, with or without amplification, is then sequenced in a high-throughput manner to obtain short sequences from one end (single-end sequencing) or both ends (pair-end sequencing). The reads are typically 30-400 bp, depending on the DNA-sequencing technology used. In principle, any high-throughput sequencing technology can be used for RNA-Seq. Following sequencing, the resulting reads are either aligned to a reference genome or reference transcripts, or assembled de novo without the genomic sequence to produce a genome-scale transcription map that consists of both the transcriptional structure and/or level of expression for each gene. To avoid artifacts and biases generated by reverse transcription direct RNA sequencing can also be applied.

Microarray: Expression levels of a gene may be assessed using the microarray technique. In this method, polynucleotide sequences of interest (including cDNAs and oligonucleotides) are arrayed on a substrate. The arrayed sequences are then contacted under conditions suitable for specific hybridization with detectably labeled cDNA generated from RNA of a test sample. As in the RT-PCR method, the source of RNA typically is total RNA isolated from a tumor sample, and optionally from normal tissue of the same patient as an internal control or cell lines. RNA can be extracted, for example, from frozen or archived paraffin-embedded and fixed (e.g., formalin-fixed) tissue samples. For archived, formalin-fixed tissue cDNA-mediated annealing, selection, extension, and ligation, DASL-Illumina method may be used. For a non-limiting example, PCR amplified cDNAs to be assayed are applied to a substrate in a dense array. Microarray analysis can be performed by commercially available equipment, following manufacturer's protocols, such as by using the Affymetrix GenChip technology, or Incyte's microarray technology.

In some embodiments, the gastrointestinal tract comprises the oral cavity. In some embodiments, the oral cavity comprises the lips, hard palate (the bony front portion of the roof of the mouth), soft palate (the muscular back portion of the roof of the mouth), retromolar trigone (the area behind the wisdom teeth), front two-thirds of the tongue, gingiva (gums), buccal mucosa (the inner lining of the lips and cheeks), and floor of the mouth under the tongue.

In some embodiments the oral cavity comprises an oral masticatory organ, comprising organs and structures primarily functioning in mastication. In some embodiments the oral cavity comprises the hard palate, gingiva, jaws or jaw muscles, teeth, temporomandibular joint, tongue, lip, cheek, or mucous membrane.

In some embodiments, the mucosal tissue comprises lamina propria. In some embodiments, obtaining is by a further step of separation of the connective tissue comprising the lamina propria, from the epithelium of the mucosal tissue. In some embodiments separation of the connective tissue from the epithelium is performed prior step (i). In some embodiments the lamina propria comprises a mesenchymal stem cell and at least one of: a fibroblast and a myofibroblast.

In some embodiments, the lamina propria comprises a fibrous connective tissue that consists of a network of type I or III collagen, or elastin fibers. In some embodiments, the main cells of the lamina propria are fibroblasts. In some embodiments, the laminal propria comprises a fibroblast, a myofibroblast, or any combination thereof. In some embodiments, the term “myofibroblast” refers to a fibroblast with characteristics of a smooth muscle cell.

In some embodiments, a characteristic of a smooth muscle cell comprises a gene expression pattern that resembles smooth muscle cells or, a phenotypic appearance of a smooth muscle cell, or any combination thereof. In some embodiments, the cell population is derived from a gastrointestinal tissue comprising fibroblasts, smooth muscle cells or any combination thereof. In some embodiments, the lamina propria tissue further comprises lymphocytes, plasma cells, macrophages, eosinophilic leukocytes, mast cells, or any combination thereof.

In some embodiments, the connective tissue of the lamina propria comprises two layers, papillary and dense:
(a) In some embodiments, the papillary layer comprises the more superficial layer of the lamina propria. In some embodiments, the papillary layer consists of loose connective tissue within the connective tissue papillae, along with blood vessels and nerve tissue.
(b) In some embodiments, the dense layer is the deeper layer of the lamina propria. In some embodiments, the dense layer consists of dense connective tissue with a larger amount of fibers, compared to the papillary layer.

In some embodiments, between the papillary layer and the deeper layers of the lamina propria is a capillary plexus. In some embodiments, the capillary plexus provides nutrition for all layers of the mucosa and sends capillaries into the connective tissue papillae.

In some embodiments, the lamina propria comprises the papillary layer, the dense layer, the capillary plexus, or any combination thereof. In some embodiments, the lamina propria comprises only one tissue layer from: the papillary layer, the dense layer, and the capillary plexus.

Techniques for separation between connective tissue of lamina propria and epithelium are known in art. Non limiting example is the use of a digestive enzyme. In some embodiments, separation is done by a dispase, (i.e., a protease that is suitable for the gentle dissociation of a wide variety of tissues). Dispase II comprises an amino-endo peptidase that hydrolyzes the N-terminal peptide bonds of non-polar amino acid residues. In some embodiments, dispase II has a mild proteolytic action that makes it useful for the isolation and routine passaging of primary cells. Other techniques include using ethylenediamine tetraacetate dihydrate (EDTA), as in Holmstrup P et al. “EDTA separation and recombination of epithelium and connective tissue of human oral mucosa.” Studies of tissue transplants in nude mice. Exp Cell Biol. 1985; 53:32-40. The contents of all of which are hereby incorporated by reference in their entirety.

In some embodiments, oral mucosa is divided into three main categories based on function or histology:
(a) masticatory mucosa, comprising keratinized stratified squamous epithelium, found on hard palate, gingiva or on the dorsum of the tongue.
(b) lining mucosa, comprising a nonkeratinized stratified squamous epithelium, found almost everywhere else in the oral cavity, including the alveolar mucosa (the lining between the buccal and labial mucosae) the buccal mucosa (the inside lining of the cheeks) the labial mucosa (the inside lining of the lips), or the mucosa lining the ventral surface of the tongue, floor of the mouth, or soft palate; and,
(c) specialized mucosa, comprising mix of masticatory and lining mucosa, found specifically in the regions of the taste buds on lingual papillae on the dorsal surface of the tongue.

In some embodiments, cell population is derived from the oral mucosa comprising masticatory mucosa, lining mucosa, specialized mucosa, and any combination thereof. In some embodiments, cell population is derived from the masticatory oral mucosa. In some embodiments, masticatory oral mucosa comprises keratinized epithelium. In some embodiments, masticatory mucosa comprises hard palate, gingiva, or the dorsal tongue surface. According to other embodiments, cells are derived from the lining oral mucosa. In some embodiments, the lining oral mucosa comprises the lips, soft palate, cheeks, or oropharynx. In the example disclosed herein, the masticatory oral mucosa comprises the main source for mesenchymal stem cells, however, as described in the example disclosed herein, mesenchymal stem cells are also found, in reduced amount, in the lining oral mucosa.

In some embodiments, cell population comprises an explant or a culture prior to a second passage. As used herein, the term “passage” is the procedure of harvesting cells from a culture, transferring the cells to one or more culture vessels with fresh growth medium, and using those cells to start new cultures. The term “passage” is also referred to as subculturing. The term “prior a second passage” comprises a culture before the first passage, i.e., prior first harvesting, or right after the first passage. Culturing conditions for enrichment of mesenchymal stem cells are known in the art. Non limiting example is described in Sotiropoulou P A et al. “Characterization of the optimal culture conditions for clinical scale production of human mesenchymal stem cells.” Stem Cells. 2006; 24:462-471. In some embodiments, culturing media comprises: dulbecco's modified eagle medium (DMEM), or DMEM with low glucose concentration (DMEM-LG) and antibiotics. In some embodiments, culturing medium further comprises about 10% foetal bovine serum. In some embodiments, media further comprises sodium pyruvate, and/or, non-essential amino acids.

In some embodiments, cell population comprises freshly isolated mesenchymal stromal cells, without culturing process.

In some embodiments, contacting is with a panel of probes, identifying a plurality of markers selected from: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2, TMEM158, or TM4SF1.

In some embodiments, plurality of markers comprises 1-3, 4-6, 7-10, 11-15, or 16-20, 21-25, 26-30, 31-35, 36-40, 41-42 of the abovementioned markers. In some embodiments, plurality of markers comprises 1-2, 3-4, 5-6, 7-8, 9-10, 11-12, 13-14, 15-16, 17-18, 19-20, 20-21, 22-23, 24-25, 26-27, 28-29, 30-31, 32-33, 34-35, 36-37, 38-39, 40-41, or 41-42 of the abovementioned markers. Each possibility represents a separate embodiment of the invention. In some embodiments, one probe targets or identifies more than one marker.

In some embodiments, identifying comprises isolation of mesenchymal stem cell subpopulation from a heterogenic population of mesenchymal stromal cells. In some embodiments, isolation is performed by cell sorting. As used herein, “cell sorting” comprises the process through which a particular cell type is separated from the others contained in a sample, based on extracellular or intracellular gene expression or protein expression. In some embodiments, the term “gene expression” comprises RNA (mRNA or other functional RNA molecule) level determination. In some embodiments, the cell is separated from the others contained in a sample, based on extracellular or intracellular protein expression. In some embodiments, cell sorting comprises separation of a particular cell based on extracellular protein expression. Methods for cell sorting are well known in art. Non limiting examples include immunomagnetic cell separation (e.g. magnetic-activated cell sorting or MACS), fluorescence-activated cell sorting (FACS), density gradient centrifugation, immunodensity cell separation, sedimentation, adhesion, microfluidic cell separation, or other techniques comprising: aptamer technology, buoyancy-activated cell sorting (BACS), complement depletion, laser capture microdissection, and immunoguided laser capture microdissection, or single-cell sorting comprising microraft array. In some embodiments, the magnetic particles in immunomagnetic cell separation are bound to a specific cell protein on the surface of a target cell by an antibody, an enzyme, lectin, or streptavidin.

In some embodiments, cell sorting comprises separation of a particular cell based on intracellular protein expression. Methods for targeting an intracellular protein by a modified antibody, nanobody, small molecule, peptide, or any other affinity reagent capable of penetrating a living cell, are well known in the art. Non limiting examples include functionalization of nanobodies that has been demonstrated in Bruce V J, Lopez-Islas M et al. Resurfaced cell-penetrating nanobodies: A potentially general scaffold for intracellularly targeted protein discovery. Protein Sci. 2016; 25:1129-1137, herein incorporated by reference in its entirety. Another example for targeting or identifying an intracellular protein in a living cell is by conjugating phosphorothioated DNA oligonucleotides to an antibody that enables its efficient cellular internalization, as demonstrated in Herrmann A et al. An effective cell-penetrating antibody delivery platform. JCI Insight. 2019; 4:e127474, herein incorporated by reference in its entirety. Another strategy for identifying intracellular protein is demonstrated in Du S et al. Cell-Permeant Bioadaptors for Cytosolic Delivery of Native Antibodies: A “Mix-and-Go” Approach. ACS Cent Sci. 2020; 6:2362-2376, herein incorporated by reference in its entirety. In some embodiments, mesenchymal stem cell subpopulation after isolation process comprises at least 50% of the heterogenic population of mesenchymal stromal cells. In some embodiments, mesenchymal stem cell subpopulation after isolation process comprises at least 50%, at least 55%, at least 60%, at least 65%, at least 70%, at least 75%, at least 80%, at least 85%, at least 90%, at least 95%, or at least 97% of the mesenchymal stromal cells. In some embodiments, mesenchymal stem cell subpopulation after isolation process comprises at least 50-54%, 55-59%, 60-64%, 65-69%, 70-74%, 75-79%, 80-84%, 85-89%, 90-94% 95-97%, or 98-99%, of the population of mesenchymal stromal cells. Each possibility represents a separate embodiment of the invention. In some embodiments, 98-100% of the cells within a sample after isolation are mesenchymal stem cells. In some embodiments, 100% of the cells within a sample after isolation are mesenchymal stem cells.

In some embodiments, isolation comprises contacting in step (ii) with at least one probe identifying a protein marker selected from: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, and PSMD2.

In some embodiments, isolation comprises contacting in step (ii) with at least one probe identifying a protein marker selected from: UBE2S, ASPM, PRC1, UBE2C, FABP5, TNFRSF12A, ODC1, NME1, DNMT1, CEP55, CKAP5, AXL, DIAPH3, PCNA, DNAJC9, ARHGAP18, NES, UAP1, LY6K, EZR, and ARHGAP22.

In some embodiments, the protein marker is intracellular. In some embodiments, the protein marker is extracellular. In some embodiments, the protein marker is on the cell surface of the cell. In some embodiments, the protein marker is membranal. In some embodiments, the protein is in an internal compartment of a cell comprising: the cytoplasm or the nucleus.

In some embodiments, an external cell surface marker comprises: TNFRSF12A, LY6K, EMP3, AXL, TMEM158, TM4SF1, or any combination thereof. In some embodiments, isolation comprises contacting the cells as disclosed herein with a panel of probes, identifying: TNFRSF12A, LY6K, EMP3, AXL, TMEM158, TM4SF1, or any combination thereof.

In some embodiments, the method further comprises a step of contacting the cells as disclosed herein with at least one probe identifying at least one marker selected from: CD44, CD73 (NT5E), CD90 (THY1), CD105 (ENG), and Stro-1. In some embodiments, contacting with at least one probe identifying at least one marker selected from: CD44, CD73 (NT5E), CD90 (THY1), CD105 (ENG), and Stro-1, is performed after step (i). Mesenchymal stem cell derived from the lamina propria of the oral mucosa are known to be positive for at least one marker, or a panel of markers selected from: CD29, CD44, CD73, CD90, CD105, CD106, CD117, CD146, CD166 and Strol+, as disclosed in WO/2008/132722-PLURIPOTENT AUTOLOGOUS STEM CELLS FROM ORAL MUCOSA AND METHODS OF USE, and in Kim D et al. Gingiva-Derived Mesenchymal Stem Cells: Potential Application in Tissue Engineering and Regenerative Medicine—A Comprehensive Review. Front Immunol. 2021; 12:667221. The contents of all of which are hereby incorporated by reference in their entirety. The present invention, in some embodiments, demonstrates that different subpopulations of mesenchymal stromal cells from the oral mucosa, comprising mesenchymal stem cells, fibroblasts or myofibroblasts, are positive, or express elevated levels, of at least one marker selected from: CD44, CD73 (NT5E), CD105 (ENG) and CD90 (THY1). In some embodiments, mesenchymal stem cells, and at least one of: fibroblasts and myofibroblasts, comprise positive expression or elevated levels of the markers: CD44, CD73 (NT5E), CD105 (ENG) CD90 (THY1), or any combination thereof.

In some embodiments, mesenchymal stem cells are positive or express elevated levels of a plurality of markers selected from: CD44, CD73 (NT5E), CD90 (THY1), CD105 (ENG), and Stro-1. In some embodiments, mesenchymal stem cells are positive or express elevated levels of 1-2, or 3-4 of the markers selected from: CD44, CD73 (NT5E), CD90 (THY1), CD105 (ENG), and Stro-1. In some embodiments, mesenchymal stem cells are positive or express elevated levels of all the markers: CD44, CD73 (NT5E), CD90 (THY1), CD105 (ENG), and Stro-1.

The CD44 antigen comprises a cell-surface glycoprotein. In some embodiments, CD44 is involved in cell-cell interactions, cell adhesion, migration, or any combination thereof. CD44 is also referred to as HCAM (homing cell adhesion molecule), Pgp-1 (phagocytic glycoprotein-1), Hermes antigen, lymphocyte homing receptor, ECM-III, or HUTCH-1. CD73 comprises a surface enzyme. In some embodiments, CD73 serves to convert AMP to adenosine. CD73 is also known as 5′-nucleotidase (5′-NT), ecto-5′-nucleotidase or NT5E. CD105 or endoglin (ENG) comprises a type I membrane glycoprotein located on cell surface and is part of the TGF beta receptor complex. CD105 is also referred to as END, FLJ41744, HHT1, ORW or ORW1. In some embodiments, CD90 comprises a 25-37 kDa heavily N-glycosylated, glycophosphatidylinositol (GPI) anchored conserved cell surface protein with a single V-like immunoglobulin domain. CD90 is also referred to as Thy-1 Cell Surface Antigen.

In some embodiments, the probe comprises an antibody, an antibody fragment, a nanobody, or a small molecule. In some embodiments, the probe comprises an enzyme, lectin, or streptavidin. In some embodiments, the probe is conjugated to detectable tracer, comprising: a fluorophore, a chromophore, a magnetic bead, or any combination thereof. In some embodiments, isolation process is performed by fluorescence activated cell sorting (FACS, or flow cytometry). In some embodiments, contacting comprises incubating the sample containing the cell population of step (i) with a probe, comprising an antibody, or an antibody fragment, that identifies at least one of the markers: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2, TMEM158, or TM4SF1, and conjugated to a fluorophore or a fluorescent dye. In other embodiments, the probe comprises a first antibody (or an antibody fragment), identifying at least one of the abovementioned markers, and contacting comprises first incubation with a first antibody, and second incubation with a secondary antibody that identifies the Fc of the first antibody, and conjugated to a fluorophore. In some embodiments, isolation comprises additional step after step (ii), comprising elution of the cells that are positive, or expressing elevated levels, of at least one of the abovementioned markers. In some embodiments, “incubation” comprises incubation in defined conditions for antigen-ligand binding (e.g., at least 10 min at RT or 4° C.).

In some embodiments, the isolation step comprises using magnetic-activated cell sorting (MACS). In some embodiments, contacting comprises incubating the sample containing the cell population of step (i) with a probe, comprising an antibody, or an antibody fragment, identifying at least one of the markers: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2, TMEM158, or TM4SF1.

In other embodiments, the probe comprises a first antibody (or an antibody fragment), identifying at least one of the abovementioned markers, and contacting comprises first incubation with a first antibody, and second incubation with a secondary antibody targeting the Fc of the first antibody and conjugated to a magnetic bead. In some embodiments, isolation comprises additional step after step (ii), comprising elution of the cells positive, or expressing elevated levels, of at least one of the abovementioned markers.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelid, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulfide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions. Immunoglobulin molecules can be of any type (e.g., IgG, IgE, IgM, IgD, IgA and IgY), class (e.g., IgG1, IgG2, IgG3, IgG4, IgA1 and IgA2) or subclass.

In some embodiments, the method comprises additional steps after isolation step, of mesenchymal stem cell in-vitro differentiation, under defined culture conditions, or of genetic introduction of specific transcription factors to the cells.

In some embodiments, the method is for treating a disorder or a disease in a subject in need thereof.

In some embodiments, the disorder or disease is selected from: a skin disorder, an autoimmune or an inflammatory disease, nerve damage, an oral or craniofacial disorder, and a bone or a cartilage disorder. In some embodiments, the method is for treating an autoimmune or an inflammatory disease. In some embodiments, mesenchymal stem cells, isolated or enriched by the method disclosed herein, have an anti-inflammatory or immunomodulatory property. In some embodiments, the mesenchymal stem cells have anti-inflammatory or immunomodulatory effect. In some embodiments, the anti-inflammatory or immunomodulatory effect is on an immune cell. Non limiting examples include: macrophages or monocytes (e.g., by polarizing macrophages to M2 phenotype), mast cells (e.g., suppressing secretion of pro-inflammatory cytokines from HMC-1 mast cell), dendritic cells, CD3+CD4+ T cells (e.g., inhibition of naïve T cells differentiation into Th1 or Th17 cells), Tregs (e.g., induction of CD4+CD25+Foxp3+Tregs), or B cells (e.g., suppressing B cell proliferation, differentiation, or antibody production). In some embodiments, a therapeutic effect is mediated by differentiation to the damaged cell lineage. In some embodiments, a therapeutic effect is mediated by differentiation to at least one of: osteoblasts, chondrocytes, adipocytes, tenocytes, myotubes, neural cells, or hematopoietic-supporting stroma. In some embodiments, the therapeutic effect comprises cell-cell contact.

In some embodiments, the therapeutic effect is mediated by a secreted factor from the mesenchymal stem cells. In some embodiments, the therapeutic effect is mediated by a secreted factor from mesenchymal stem cells-derived differentiated cells.

In some embodiments, the disorder or disease comprises a skin disorder, comprising skin wound, skin allografts, skin contact hypersensitivity, or psoriasis. In some embodiments, the disorder or disease comprises an autoimmune or an inflammatory disease. In some embodiments, the autoimmune disease or the inflammatory disease comprises colitis, arthritis, graft versus host disease (GvHD), type 1 diabetes mellitus (T1DM), lupus nephritis, osteoporosis, atherosclerosis, or bone marrow failure (e.g., human aplastic anemia, manifested by severe pancytopenia and bone marrow failure). In some embodiments, the autoimmune disease comprises addison disease, celiac disease-sprue (gluten-sensitive enteropathy), dermatomyositis, graves' disease, hashimoto thyroiditis, multiple sclerosis, myasthenia gravis, pernicious anemia, reactive arthritis, rheumatoid arthritis, sjögren syndrome, guillain-barre syndrome, chronic inflammatory demyelinating polyneuropathy (CIDP), systemic lupus erythematosus, psoriasis, graves' disease, inflammatory bowel disease, scleroderma, vasculitis, or type I diabetes.

In some embodiments, the disorder comprises oral or craniofacial disorder, comprising a periodontal disease, peri-implantitis, maxillofacial bone defect, calvarial bone defect, palatal defect, gingival defect, oral mucositis, tongue defect, craniofacial muscle damage, or facial nerve damage. In some embodiments, the disorder or disease comprises nerve damage comprising facial nerve damage, sciatic nerve damage, or spinal cord injury. In some embodiments, the disorder or disease comprises a bone defect or injury. In some embodiments, the disorder comprises cartilage defect or injury.

It should be noted that the therapeutic properties of mesenchymal stem cells, in general, and particularly oral mucosa-derived mesenchymal stem cells, are well known in the art. Thus, in some embodiments, any disease or disorder that benefits from therapy with mesenchymal stem cell, mesenchymal stem-derived differentiated cell, or mesenchymal stem cell-derived secreted factor, may be treated with the method disclosed herein. Non-limiting examples of diseases that can be treated by utilizing mesenchymal stem cell derived from the gastrointestinal tube include: bone fractures, osteoporosis, ligament rupture, osteoarthritis, any arthritis of autoimmune origin, traumatic articular cartilage damage, myocardial infarction, heart failure, mitral or aortic valves insufficiency, coronary insufficiency, diabetes, hepatic insufficiency or failure, reflux, fecal incontinence, urinary incontinence, renal insufficiency or failure, emphysema, Parkinson disease, muscular atrophies, muscular dystrophies, amyotrophic lateral sclerosis, multiple sclerosis and other demyelinating diseases, myasthenia gravis, polymyositis, loss of brain tissue caused by cerebrovascular diseases or encephalitis or meningitis, insufficiency and failure of endocrine glands (e.g. hypothyroidism, hypoparathyroidism, pituitary and adrenal hypofunction), acquired or induced failure of the hematopoietic system, periodontal disease and loss of any other tissue mass and function caused by degenerative, inflammatory, proliferative, infectious, malignant diseases, trauma and aging.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

In some embodiments, the isolated mesenchymal stem cells are administered to the same subject, from which the gastrointestinal tissue was derived (i.e., autologous transplantation). In other embodiments, the isolated mesenchymal stem cells are administered to a different subject, from which the gastrointestinal tissue was derived (i.e., allogeneic transplantation).

As used herein, the terms “administering”, “administration” and like terms refer to any method which, in sound medical practice, delivers a composition containing an active agent to a subject in such a manner as to provide a therapeutic effect.

As used herein, the terms “subject” or “individual” or “animal” or “patient” or “mammal” refers to any subject, particularly a mammalian subject, for whom therapy is desired, for example, a human.

### Kits

According to some embodiments, there is provided a kit for identification of a multipotent mesenchymal stem cell subpopulation, among a population of cells. In some embodiments, the kit comprises at least one probe, capable of identifying at least one marker, selected from: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2, TMEM158, TM4SF1, or any combination thereof.

In some embodiments the kit comprises at least two probes, capable of identifying at least two markers from the abovementioned markers. In some embodiments, the kit comprises 1-3, 4-6, 7-10, 11-15, or 16-20, 21-25, 26-30, 31-35, 36-40, 41-42 of probes, capable of identifying 1-3, 4-6, 7-10, 11-15, or 16-20, 21-25, 26-30, 31-35, 36-40, 41-42 of the markers. In some embodiments, plurality of markers comprises 1-2, 3-4, 5-6, 7-8, 9-10, 11-12, 13-14, 15-16, 17-18, 19-20, or 20-21, 22-23, 24-25, 26-27, 28-29, 30-31, 32-33, 34-35, 36-37, 38-39, 40-41, or 41-42 markers. Each possibility represents a separate embodiment of the invention.

In some embodiments, there population of cells comprises a heterogenic population of gastrointestinal tract mucosa mesenchymal stromal cells.

In some embodiments, the kit comprises:
(a) at least one probe, targeting at least one marker, selected from UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2, TMEM158, or TM4SF1; and,
(b) at least one probe targeting at least one marker selected from: CD44, CD73 (NT5E), CD90 (THY1), CD105 (ENG), or Stro-1.

In some embodiments, the probe within the kit comprises an antibody, or an antibody fragment, conjugated to detectable tracer, comprising: a fluorophore, a chromophore, or a magnetic bead. In some embodiments, the antibody comprises a fluorescent dye-conjugated antibody. In some embodiments, the kit comprises at least one reagent for FACS cell sorting.

In some embodiments, at least one reagent comprises a fixation buffer, a permeabilization buffer, an elution buffer, or a first antibody (directly identifying the marker) or a secondary antibody (identifying the Fc of the first antibody)-conjugated to fluorophore. In some embodiments, the antibody comprises a magnetic bead-conjugated antibody. In some embodiments, the kit comprises at least one reagent for MACS sorting. In some embodiments, at least one reagent comprises a first antibody or a secondary antibody-conjugated to a magnetic bead.

### Pharmaceutical Compositions

In some embodiments, there is provided a composition comprising an enriched or isolated mesenchymal stem cell subpopulation and an acceptable carrier, wherein the subpopulation:
(a) comprises at least 50% of the mesenchymal stromal cells,
(b) characterized by above a predetermined threshold expression of at least one marker, selected from: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2, TMEM158, TM4SF1, or any combination thereof. In some embodiments, the enriched or isolated mesenchymal stem cell is derived from heterogenic mesenchymal stromal cell explant or culture, derived from the gastrointestinal tract mucosa.

In some embodiments, at least 50% of the mesenchymal cells are mesenchymal stromal cells.

In some embodiments, the mesenchymal stem cell comprises a multipotent phenotype. In some embodiments, the mesenchymal stem cell comprises an anti-inflammatory or an immunomodulatory capacity. In some embodiments, the mesenchymal stem cell comprises an ability to secrete a therapeutic factor, i.e., a factor comprising a protein or RNA molecule (e.g., miRNA), with a therapeutic effect, in a subject in need thereof.

In some embodiments, the pharmaceutical composition comprises the product of the method disclosed herein. In some embodiments, mesenchymal stem cell subpopulation within the pharmaceutical composition comprises at least 50% of the heterogenic population of mesenchymal stromal cells. In some embodiments, mesenchymal stem cell subpopulation comprises at least at least 50%, at least 55%, at least 60%, at least 65%, at least 70%, at least 75%, at least 80%, at least 85%, at least 90%, at least 95%, or at least 97% of the mesenchymal stromal cells. In some embodiments, mesenchymal stem cell subpopulation within the pharmaceutical composition comprises at least 50-54%, 55-59%, 60-64%, 65-69%, 70-74%, 75-79%, 80-84%, 85-89%, 90-94% 95-97%, or 98-99%, of the heterogenic population of mesenchymal stromal cells. Each possibility represents a separate embodiment of the invention. In some embodiments, 98-100% of the cells within the pharmaceutical composition are mesenchymal stem cells. In some embodiments, 100% of the cells within the pharmaceutical composition are defined as mesenchymal stem cells.

In some embodiments, the carrier within the pharmaceutical composition is dependent on the route of administration. In some embodiments, stem cell route of administration comprises intravenous, intracerebral, intraventricular, subcutaneous, subarachnoid, intra-arterial, intraperitoneal, intranasal, intramuscular or by direct intra-tissue injection. In some embodiments, the pharmaceutical composition is administrated intravenous (IV). In some embodiments, the carrier comprises IV acceptable carrier, excipient, or a diluent. In some embodiments, the carrier comprises an isotonic saline.

In some embodiments, a multipotent phenotype of a mesenchymal stem cell within the pharmaceutical composition is defined as the cell ability to differentiate into more than one lineage. In some embodiments, the multipotent phenotype of a mesenchymal stem cell within the composition comprises an ability to differentiate into osteogenic lineage.

In some embodiments, the term “above a predetermined threshold expression” comprises at least 1.2-fold, 1.3-fold, 1.4-fold, 1.5-fold, 2-fold, 3-fold, 4-fold 5-fold, 6-fold, 7-fold, 8-fold, 9-fold, or 10-fold induction in the expression. Each possibility represents a separate embodiment of the invention.

In some embodiments, there is provided a method for treating an individual suffering from a disorder or a disease, in need thereof, with the pharmaceutical composition disclosed herein.

In some embodiments, a therapeutically effective dose of the composition of the invention is administered. In some embodiments, “a therapeutically effective dose” is determined according to the disease or disorder. In some embodiments, “a therapeutically effective dose” is determined according to the route of administration. In some embodiments, the median dose for IV delivery is about 100 million MSCs/subject/dose. In some embodiments, the dose for IV injection comprises 50 to 250 million MSCs/subject/dose. In other embodiments, the pharmaceutical composition dose comprises 10-50 million MSCs/subject/dose.

The term “therapeutically effective amount” refers to an amount of a drug effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

The dosage administered will be dependent upon the age, health, and weight of the recipient, kind of concurrent treatment, if any, frequency of treatment, and the nature of the effect desired. The route of administration of the pharmaceutical compositions will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art.

As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S.

Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21^{st} Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum.

Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

As used herein, the term “about” when combined with a value refers to plus and minus 10% of the reference value. For example, a length of about 1000 nanometers (nm) refers to a length of 1000 nm±100 nm.

It is noted that as used herein and in the appended claims, the singular forms “a”, “an”, and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Isolation and Culture of Primary Human Oral Mucosa-Derived Mesenchymal Stromal Cells

The experiments were approved by the Tel Aviv University institutional review board (IEC No. 0003692-1). Informed consent was obtained from all patients. Cells were isolated and cultured as previously described 8. Briefly, fragments of masticatory mucosa (from the posterior hard palate) and oral lining mucosa (from the alveolar mucosa of the anterior mandible) were harvested from three female Caucasian donors, aged 20-30 years, during periodontal mucogingival procedures. Shallow tissue samples (without submucosa) with a diameter of approximately 2 mm were excised using a #15C scalpel blade. Exclusion criteria were smoking, any systemic disease, pregnancy and lactation, or a history of periodontal disease. Tissue fragments were separated into connective tissue and epithelium by a 2 h—incubation with dispase II (2 mg/ml, Sigma-Aldrich, Rehovot, Israel) at 37° C. The connective tissue was cut into smaller pieces, which were then incubated in standard culture medium (SCM, Dulbecco's Modified Eagle Medium (DMEM) with 10% fetal calf serum [FCS], 2 mM glutamine, 100 U/ml penicillin, 100 mg/ml streptomycin, 12.5 U/ml nystatin, 0.11 mg/ml sodium pyruvate, and non-essential amino acids [Biological Industries, Beit HaEmek, Israel]) at 37° C. in a humidified atmosphere of 5% CO2 and 95% air, to allow for cell outgrowth. The medium was replaced every 3 days until confluence was reached.

Cell viability was assessed manually by trypan blue dye exclusion (Biological Industries). Only first passage cells with a typical fibroblastic morphology and cell viability levels >95%, were used for scRNA-seq.

### Osteogenic Differentiation and Staining

Second or third passage cells were seeded at 5×10³ cells per well in 96-well plates in quadruplicates and were allowed to attach for 48 h in SCM. They were then cultured in an osteogenic medium [SCM+0.1M ascorbic acid and 0.1M β-glycerophosphate (Sigma-Aldrich, Israel)], which was changed every 2 days. After 21 days, the cells were fixed in a 1:1:1.5 solution of 10% formalin/methanol/water for 2 h, washed with distilled water, and stained with Alizarin Red (Sigma-Aldrich, Israel) for 30 minutes.

### Single-Cell RNA-Seq Library Preparation & Sequencing

Cells at 60-70% confluence were harvested, washed, and resuspended in PBS supplemented with 0.5% BSA to achieve an optimal concentration of approximately 1000 cells per microliter for loading onto the Next GEM Chip (10× Genomics). Libraries were prepared using the 10× Genomics Chromium Controller in conjunction with the single-cell 3′ v3.1 kit, protocol revision D. The cDNA synthesis, barcoding, and library preparation were carried out according to the manufacturer's instructions. Briefly, cDNA amplification was performed for 11 cycles. Sample index PCR was performed for 12 cycles using Chromium i7 Sample Indices. The resulting libraries were quantified and analyzed by Qubit and TapeStation, and were sequenced on the NextSeq™ 500 platform (Illumina), following the manufacturer's protocol, and a NextSeq 500/550 High Output Kit v2.5 (75 Cycles) kit (Illumina).

### Single Cell RNA-Seq Analysis

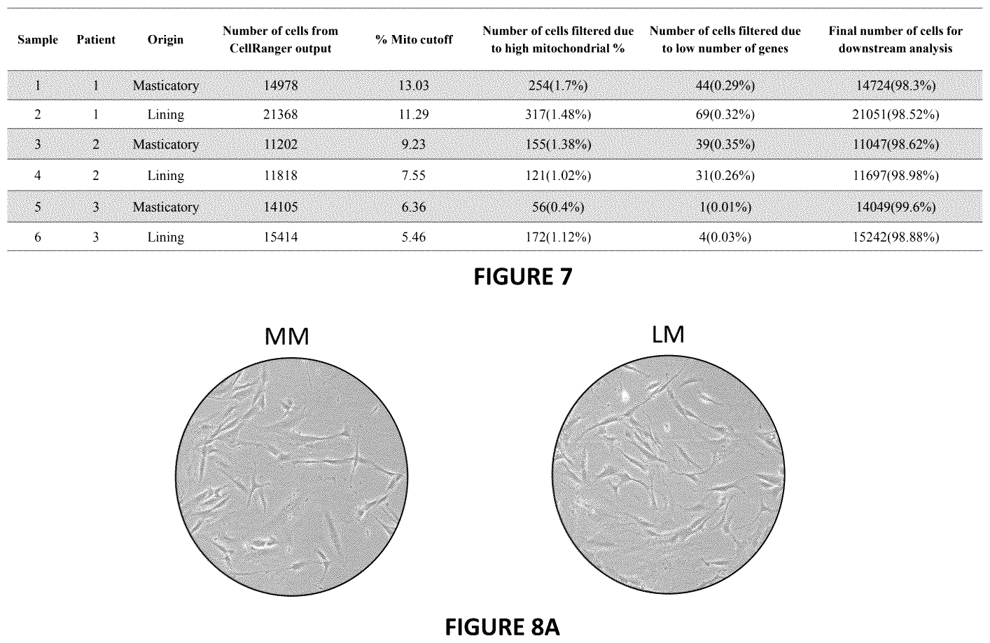

CellRanger pipeline (v6.0.1, 10× Genomics) with default parameters was used for demultiplexing, alignment (hg38 reference genome, 2020-A version, downloaded from 10× Genomics website), filtering, barcode counting, and Unique Molecular Identifier (UMI) counting. The Seurat package in R (v4.0.4) was used for downstream analysis and visualization. Gene-cell matrices were filtered to remove cells with 4 median absolute deviations (MAD) above the median of mitochondrial genes of each sample and with less than 250 genes and 500 UMIs. In addition, genes expressed by less than ten cells were removed from the analysis. After quality control, a total of 87,810 cells (from all samples) and 26,372 genes were retained, as disclosed in FIG. 7. The integration method from Seurat was used to merge the samples into one dataset according to the patient and to compensate for batch effects. Briefly, the counts of each sample were log normalized using the NormalizeData function and the 2000 most variable genes from each sample were identified using the FindVariableFeatures function with the “vst” method. The FindIntegrationAnchors function was used to identify anchors between the samples and an integrated assay was created using the IntegrateData function. Each cell was assigned a cell-cycle score using the CellCycleScoring function and the G2/M and S phase markers lists from the Seurat package. The integrated dataset was then scaled with the ScaleData function. The cell cycle scores were regressed out during the scaling process. Scaled genes with the highest variance (using the “vst” method) were used to perform linear dimensional reduction (principal component analysis). Seurat's unsupervised graph-based clustering and uniform manifold approximation (UMAP) were conducted on the projected principal component (PC) space using 23 PCs. The distinct clusters were annotated using SingleR. The annotations were taken from Human Primary Cell Atlas (biogps.org/dataset/BDS_00013/primary-cell-atlas). Marker genes were found by analyzing the differential expression based on the non-parametric Wilcoxon rank sum test (Seurat's default FindMarkers). Visualization used Seurat's FeaturePlot, DotPlot and VlnPlot functions.

### Gene Ontology (GO) Enrichment Analysis

The ClusterProfiler package in R was used for functional enrichment analysis using the molecular functions and biological pathways GO annotations and Kyoto Encyclopedia of Genes and Genomes (KEGG) pathways. The enrichment addressed the marker list of each cluster.

### Differential Expression Analysis Using a Semi-Bulk Count Matrix to Simulate Bulk RNA-Seq Data

For differential expression (DE) analysis, the counts of all the cells from each origin were aggregated to create a semi-bulk count matrix. The analysis was performed using the DESeq2 package in R with the betaPrior, cooksCutoff and independentFiltering parameters set to False. The design matrix included patient origin in addition to the case-control status. Raw P values were adjusted for multiple testing using the procedure of Benjamini and Hochberg. Differentially expressed genes were determined by a p-adj of <0.05, an absolute fold change >2, and a count of at least 30 in one sample.

### Example 1

### Cell Characterization Based on Canonical Mesenchymal Stromal Cell Features

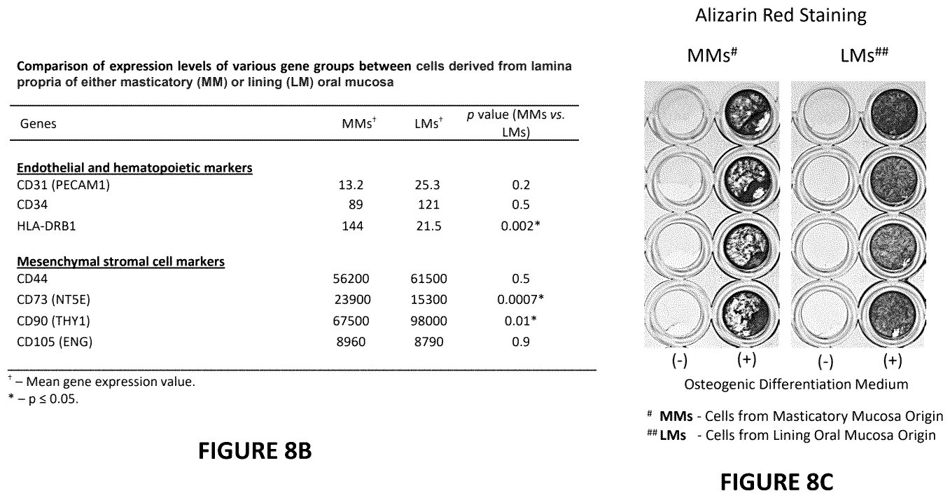

Assays based on the criteria published by the ISCT® were used to confirm the presence of mesenchymal stromal cell features in the oral mucosae-derived cells (Viswanathan, S. et al. “Mesenchymal stem versus stromal cells: International Society for Cell & Gene Therapy (ISCT®) Mesenchymal Stromal Cell committee position statement on nomenclature”. Cytotherapy 2019; 21:1019-1024, herby incorporated by reference in its entirety). All subcultured lamina propria cells, whether derived from masticatory or lining oral mucosa, maintained their adherence to plastic and displayed a similar elongated, spindle-shaped morphology, regardless of passage number (FIG. 8A). In addition, all the cells displayed high average expression levels of specific mesenchymal stromal cell markers (CD44, CD73 (NT5E), CD90 (THY1), and CD105 (ENG), while lacking hematopoietic and endothelial markers (CD31 (PECAM1), CD34, and HLA-DR), as evaluated by DE analysis of the semi-bulk count matrix (FIG. 8B). Finally, both types of cells demonstrated the ability to differentiate into mineralized matrix-producing osteoblasts in an in vitro differentiation system (FIG. 8C).

### Example 2

### Differential Expression Analysis Using a Semi-Bulk Count Matrix to Simulate Bulk RNA-Seq Data

The inventors characterized the unique features of each cell type, by DE analysis using a semi-bulk count matrix. Cells derived from the lamina propria of the masticatory mucosa exhibited statistically significant differences in specific gene groups and pathways from those derived from the lining oral mucosa. These results are in full agreement with the inventors previously published bulk RNA-seq data, and include genes for extracellular components like structural collagens and elastin, cranial neural crest markers, and homeobox genes, all of which were expressed at higher levels in lining oral mucosa cells (Table 1). Interestingly, the lining oral mucosa cells also exhibited higher levels of elastase (Table 1), suggesting a higher turnover rate of elastin compared to masticatory mucosa derived cells.

TABLE 1
Comparison of the expression levels (mean
gene expression value) of various gene groups
between cells derived from lamina propria of either
masticatory (MM) or lining (LM) oral mucosa.

| | | | |
|---|---|---|---|
| | | | p value |
| Genes | MM | LM | (MMs Vs. LMs) |

Structural collagens

| | | | |
|---|---|---|---|
| COL1A1 | 317000 | 487000 | *0.0002 |
| COL1A2 | 401000 | 588000 | *0.001 |
| COL3A1 | 39600 | 130000 | *0 |
| COLAA5 | 54.2 | 579 | *0 |
| COL4A6 | 1.6 | 29.7 | *0.002 |
| COL5A1 | 8540 | 19800 | *0.00001 |
| COL5A2 | 21400 | 41300 | *0 |

Matricellular proteins

| | | | |
|---|---|---|---|
| SPARC | 93500 | 162000 | *0.02 |
| THBS2 | 10500 | 16400 | 0.25 |

Cranial neural crest markers

| | | | |
|---|---|---|---|
| LHX8 | 7610 | 10300 | *0.008 |
| PAX3 | 1860 | 9260 | *0 |
| TFAP2A | 1520 | 4990 | 0.051 |

Positional memory markers

| | | | |
|---|---|---|---|
| HOXB2 | 12.5 | 31.3 | 0.7 |
| HOXD8 | 4.8 | 539 | *0 |

Elastin and Elastase

| | | | |
|---|---|---|---|
| ELN (Elastin) | 5760 | 11300 | *0.008 |
| ELANE (Elastase) | 197 | 617 | *0.000002 |

*p ≤ 0.05.

### Example 3

### Single-Cell RNA-Seq Analysis of Oral Mucosa-Derived Mesenchymal Stromal Cells

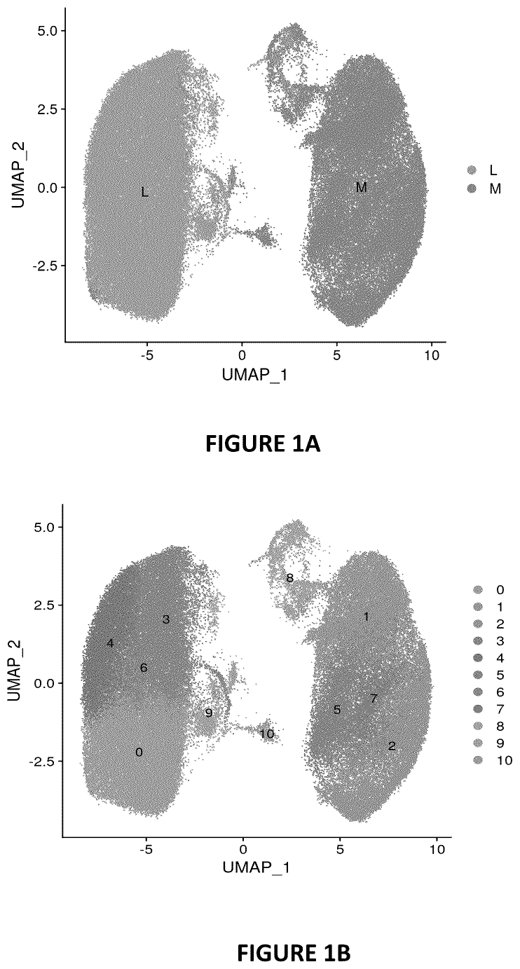

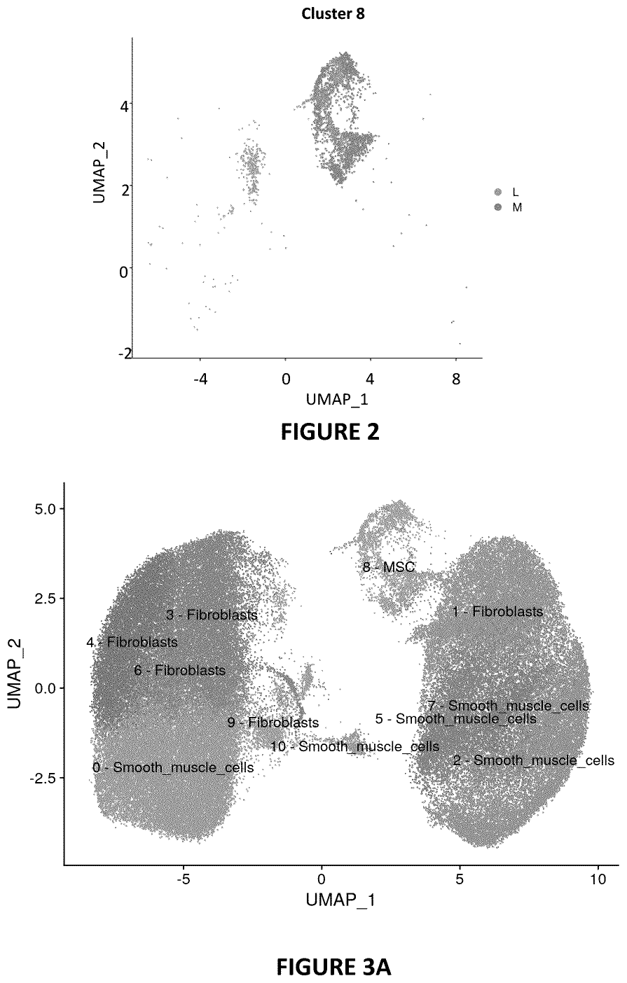

Uniform manifold approximation and projection (UMAP) visualization could clearly distinguish between the masticatory and lining oral mucosa origin of the mesenchymal stromal cells derived from the lamina propria of three individuals since the two cell types formed two clear aggregates, with minimal overlap (FIG. 1A). In contrast, there was a significant overlap of the aggregates of the corresponding tissue samples from the different individuals (data not shown), indicating that single-cell variability is predominantly the result of tissue identity, and not interpersonal variability. Cluster analysis revealed 11 distinct cell clusters or subpopulations (FIG. 1B), where clusters #0, 3, 4, 6, and 9 primarily contain cells derived from the lamina propria of lining oral mucosa, while clusters #1, 2, 5, 7, and 10 contain mainly cells of masticatory mucosal origin. The only considerable overlap between the two types of cells was found in cluster #8, which is located at the intersection of the two tissues (FIG. 2).

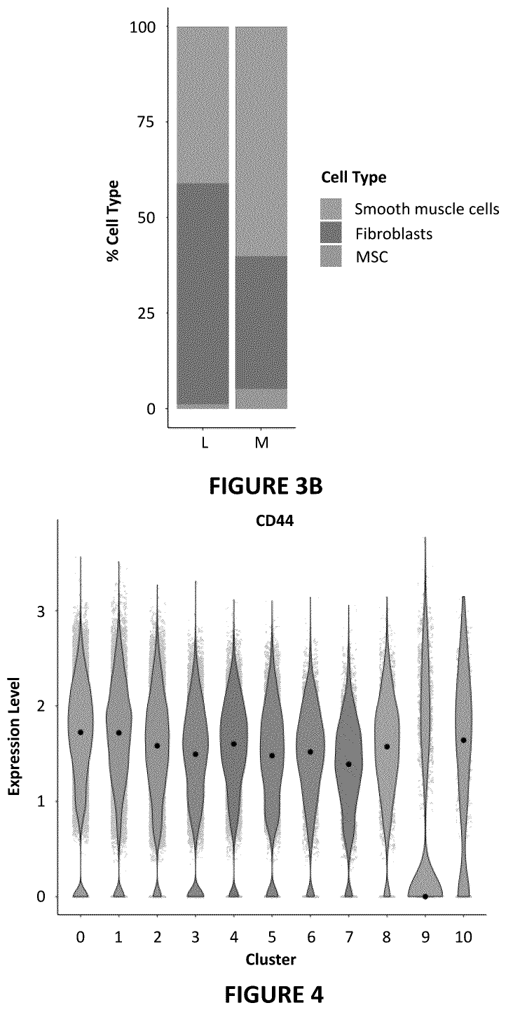

The use of Seurat allowed to identify differentially expressed gene (DEG) markers that define each cluster. When these cell clusters were annotated using SingleR (FIG. 3A), some (#1, 3, 4, 6, 9) were identified as fibroblasts and others (#0, 2, 5, 7, 10) as smooth muscle cells. The majority of cells derived from masticatory mucosa (clusters #2, 5, 7, FIGS. 3A and 3B) exhibited a smooth muscle cell-like gene expression pattern, while most cells from the lining oral mucosa (clusters #3, 4, 6, 9) were characterized by a fibroblast-like gene expression pattern. Notably, cluster #8 (at the intersection of the lining and masticatory mucosa-derived cells) was annotated as MSCs. Most members of this cluster (78%) were cells derived from the lamina propria of the masticatory mucosa.

Interestingly, nearly all cell clusters, including cluster #8, annotated as MSCs, exhibited high expression levels of typical mesenchymal stromal cell markers CD44, CD73 (NT5E), CD90 (THY1), and CD105 (ENG) (FIG. 4). This demonstrates that this criterion is unable to identify MSCs from the bulk population of oral mucosa-derived mesenchymal stromal cells, and supports the position of the ISCT® that the interchangeable use of the terms “mesenchymal stem cells” and “mesenchymal stromal cells” is inaccurate.

### Example 4

### Gene Ontology Enrichment Analysis

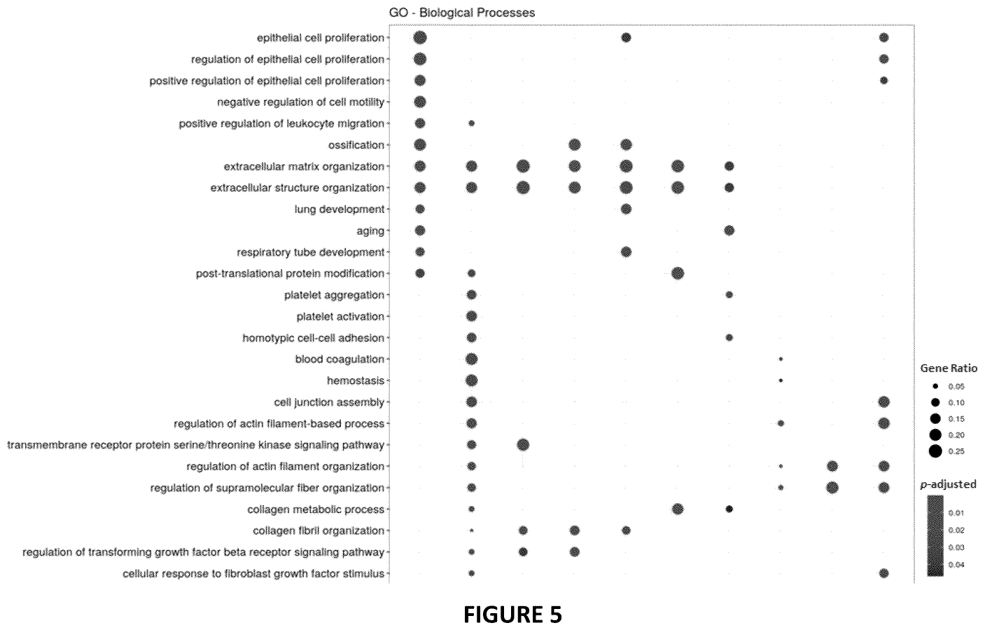

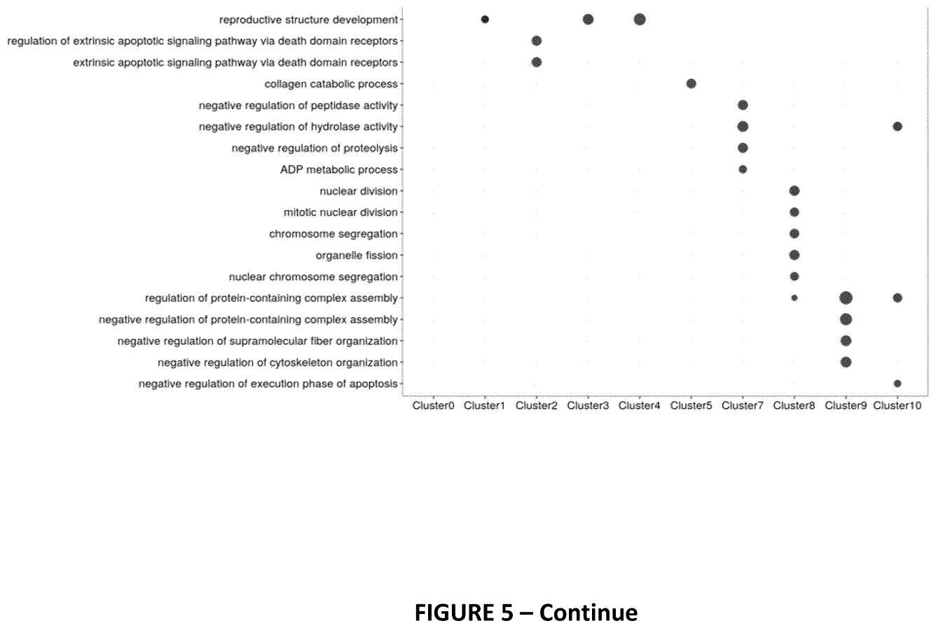

The identified cluster-specific markers were subjected to GO and KEGG enrichment analysis with the ClusterProfiler package in R (FIG. 5) in order to investigate the biological processes and signaling pathways associated with each cluster. The results indicated that each cluster is enriched for distinct biological processes and pathways, with only a moderate overlap between them. Common members include mainly extracellular matrix components: “extracellular matrix organization” (e.g., FBLN1, MFAP4, and Col3A1), “extracellular structure organization” (e.g., VCAN, DCN, and TGFBI) and “collagen fibril organization” (e.g., GREM1, Coll2A1, and ANXA2) (FIG. 5).

Interestingly, cell cluster #0 (consisting predominantly of lining oral mucosa cells) was highly enriched for biological processes associated with the regulation of epithelial cells, such as “epithelial cell proliferation” (e.g., CXCL12, IGFBP5 and FGF7), “regulation of epithelial cell proliferation” (e.g., FGF7, OSR2 and PGF) and “positive regulation of epithelial cell proliferation” (e.g., PGF, MDK and PTN). Notably, only a small proportion of masticatory mucosa cells (cluster #10) demonstrated some degree of enrichment for these processes.

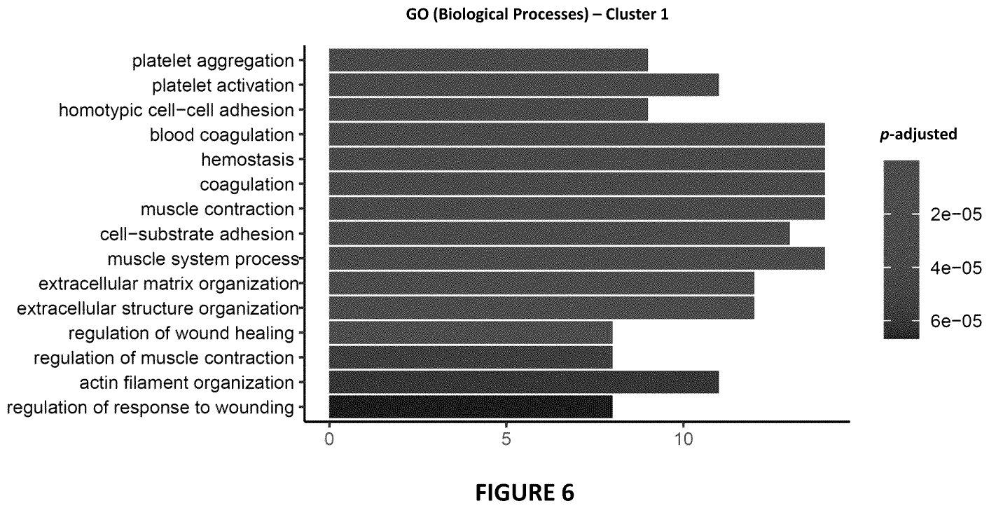

In contrast, cluster #1 (consisting mainly of cells of masticatory mucosa origin) was highly enriched for biological processes associated with wound healing, such as “regulation of wound healing” (e.g., THBS1, ANXA1, and CAV1), “regulation of response to wounding” (e.g., SERPINGI, PLPP3, and MDK), “positive regulation of wound healing” (e.g., ANXA1, MYLK, and F2R) and “positive regulation of response to wounding” (e.g., PLPP3, MDK, and PTN) (FIG. 6). Furthermore, the cells forming cluster #1 showed upregulation of POSTN, which is essential for tissue repair, and TNFRS11B (osteoprotegerin), which is a key regulator of osteoclast activity and thus a negative effector of bone resorption.

Cells in clusters #2-7 were primarily characterized by common biological processes, including mainly extracellular matrix components: “extracellular matrix organization” and “extracellular structure organization” (FIG. 5).

Cells in clusters #9-10 were primarily enriched with genes involved in regulation of actin filament organization (e.g., TPM1, CFL1 and FLNA) and supramolecular fiber organization (e.g., MYADM, TMOD3 and COTL1).

Despite adjustment for cell cycle, the cells of cluster #8, annotated as MSCs, presented a strong proliferation signature and exhibited high expression of genes related to DNA replication and cell cycle progression. Accordingly, cluster #8 was highly enriched for biological processes associated with proliferation, such as “nuclear division” (e.g., CENPF, PTTG1, and TOP2A), “mitotic nuclear division” (e.g., CENPF, MKI67, and CCNB1) and “chromosome segregation” (e.g., CCNB1, PRC1, and NUSAP1). However, cells in cluster #8 were also enriched with stemness-associated genes unrelated to the cell cycle, including NES (perivascular progenitor marker), proteasome complex subunits (PSMC3, PSMD2, PSMD14, and PSME2), which play pivotal roles in the regulation of self-renewal, pluripotency, and differentiation of stem cells, and DNMT1, which participates in self-renewal. An additional marker, HMGB2, which is associated with negative regulation of apoptosis was also strongly co-expressed in cluster #8 cells, thereby supporting the connection with self-renewal. In support, gene ontology analysis confirmed that cells in cluster #8 were enriched for markers characteristic of biological processes associated with stemness: “regulation of stem cell differentiation” (e.g., PSMD2, PSME2, and PSMC3) and “stem cell differentiation” (e.g., CFL1, CORO1C, and PSMA7). Together, these results demonstrate that the specific subpopulation of ex vivo-expanded oral mucosa-derived mesenchymal stromal cells in cluster #8 possesses an MSC-like gene expression profile.

In another experiment, single-cell RNA-seq analysis was performed on 6 different samples merely from the oral masticatory mucosa. Table 2 summarizes the top genes that were found to be a least 1.4-fold upregulated (Log₂FC higher that 0.5), within the mesenchymal stem cell cluster, compared to the rest of the clusters.

Table 3 ranks the top genes that were above the threshold of 1.4-fold induction, according their specifity, as calculated by pct.1 (percentage of expression by mesenchymal stem cells) divided by pct.2 (percentage of expression by mesenchymal non-stem cells), whereas table 4 ranks the genes according to their fold-upregulation ratio. As can be seen, UBE2S, ASPM, PRC1, UBE2C, FABP5, TNFRSF12A, ODC1, NME1, DNMT1, CEP55, CKAP5, AXL, DIAPH3, PCNA, DNAJC9, ARHGAP18, NES, UAP1, LY6K, EZR, and ARHGAP22 are among the top-ranked genes, according to both specifity, and fold-upregulation parameters.

TABLE 2
Summary of genes significantly induced in mesenchymal
stem cell subpopulation, by single-cell RNA-seq analysis.

| Average | | | Adjusted | |
|---|---|---|---|---|
| Log2FC | pct.1 | pct.2 | p-value | Gene name |
| 1.913895181 | 0.863 | 0.459 | 0 | UBE2S |
| 1.47306618 | 0.584 | 0.064 | 0 | ASPM |
| 1.418643072 | 0.715 | 0.083 | 0 | PRC1 |
| 1.346902311 | 0.502 | 0.046 | 0 | UBE2C |
| 1.31631462 | 0.822 | 0.229 | 0 | FABP5 |
| 1.18588711 | 0.952 | 0.548 | 0 | TNFRSF12A |
| 1.172937553 | 0.983 | 0.789 | 0 | S100A16 |
| 1.14266379 | 0.879 | 0.4 | 0 | ODC1 |
| 1.140023006 | 0.995 | 0.884 | 0 | ENO1 |
| 1.082664245 | 0.96 | 0.621 | 0 | NME1 |
| 0.956597847 | 0.542 | 0.034 | 0 | CEP55 |
| 0.85693072 | 0.594 | 0.116 | 0 | DIAPH3 |
| 0.808587125 | 0.999 | 0.989 | 0 | YBX1 |
| 0.764243659 | 0.953 | 0.756 | 0 | C1QBP |
| 0.743048627 | 0.962 | 0.735 | 0 | EIF5A |
| 0.733346057 | 0.577 | 0.124 | 0 | DNAJC9 |
| 0.67593576 | 0.776 | 0.414 | 0 | ARHGAP18 |
| 0.669964326 | 0.773 | 0.447 | 0 | UAP1 |
| 0.66116738 | 0.965 | 0.813 | 0 | ARPC1A |
| 0.655425509 | 0.501 | 0.26 | 0 | LY6K |
| 0.645928039 | 0.601 | 0.188 | 0 | EZR |
| 0.638945788 | 0.613 | 0.206 | 0 | ARHGAP22 |
| 0.618332583 | 0.935 | 0.726 | 0 | PDAP1 |
| 0.586589091 | 0.996 | 0.965 | 0 | EMP3 |
| 0.558694007 | 0.989 | 0.939 | 0 | CAVIN3 |
| 0.541793234 | 0.916 | 0.711 | 0 | PSMC5 |
| 0.541471309 | 0.947 | 0.79 | 0 | CCT3 |
| 0.531743367 | 0.796 | 0.5 | 0 | COMMD4 |
| 0.530389838 | 1 | 0.997 | 0 | ANXA2 |
| 0.517804887 | 0.883 | 0.663 | 0 | PSMD9 |
| 0.517266737 | 0.907 | 0.694 | 0 | COPRS |
| 0.505095869 | 0.698 | 0.433 | 0 | PALM2- |
| | | | | AKAP2 |
| 0.907767776 | 0.857 | 0.293 | 0 | CKAP5 |
| 0.523556833 | 0.915 | 0.617 | 1E−159 | CDC42EP3 |
| 0.85774681 | 0.887 | 0.54 | 0 | AXL |
| 0.54079305 | 0.838 | 0546 | 5.896E−265 | MSN |
| 0.61318373 | 0.827 | 0.47 | 0 | WDR1 |
| 1.002471985 | 0.827 | 0.354 | 0 | DNMT1 |
| 1.118138997 | 0.667 | 0.444 | 0 | HMGB2 |
| 0.670133823 | 0.69 | 0.291 | 0 | NES |
| 0.835941364 | 0.661 | 0.256 | 0 | PCNA |
| 0.682105894 | 0.912 | 0.643 | 0 | PSMD2 |
| 1.13783359 | 0.683 | 0.252 | 0 | TMEM158 |
| 1.399198634 | 0.528 | 0.099 | 0 | TM4SF1 |

TABLE 3
Specificity of mesenchymal stem cell markers as examined by pct.1
(% expression by mesenchymal stem cells) divided by pct.2
(% expression by mesenchymal non-stem cells).

| | pct.1/pct.2- MSCs specificity | Marker |
|---|---|---|
| 1 | 15.94117647 | CEP55 |
| 2 | 10.91304348 | UBE2C |
| 3 | 9.125 | ASPM |
| 4 | 8.614457831 | PRC1 |
| 5 | 5.333333333 | TM4SF1 |
| 6 | 5.120689655 | DIAPH3 |
| 7 | 4.653225806 | DNAJC9 |
| 8 | 3.589519651 | FABP5 |
| 9 | 3.196808511 | EZR |
| 10 | 2.975728155 | ARHGAP22 |
| 11 | 2.924914676 | CKAP5 |
| 12 | 2.710317460 | TMEM158 |
| 13 | 2.58203125 | PCNA |
| 14 | 2.371134021 | NES |
| 15 | 2.336158192 | DNMT1 |
| 16 | 2.1975 | ODC1 |
| 17 | 1.926923077 | LY6K |
| 18 | 1.880174292 | UBE2S |
| 19 | 1.874396135 | ARHGAP18 |
| 20 | 1.759574468 | WDR1 |
| 21 | 1.737226277 | TNFRSF12A |
| 22 | 1.729306488 | UAP1 |
| 23 | 1.642592593 | AXL |
| 24 | 1.612009238 | PALM2-AKAP2 |
| 25 | 1.592 | COMMD4 |
| 26 | 1.54589372 | NME1 |
| 27 | 1.534798535 | MSN |
| 28 | 1.502252252 | HMGB2 |
| 29 | 1.482982172 | CDC42EP3 |
| 30 | 1.418351477 | PSMD2 |
| 31 | 1.331825038 | PSMD9 |
| 32 | 1.308843537 | EIF5A |
| 33 | 1.306916427 | COPRS |
| 34 | 1.288326301 | PSMC5 |
| 35 | 1.287878788 | PDAP1 |
| 36 | 1.260582011 | C1QBP |
| 37 | 1.245880862 | S100A16 |
| 38 | 1.198734177 | CCT3 |
| 39 | 1.18696187 | ARPC1A |
| 40 | 1.125565611 | ENO1 |
| 41 | 1.053248136 | CAVIN3 |
| 42 | 1.032124352 | EMP3 |
| 43 | 1.010111223 | YBX1 |
| 42 | 1.003009027 | ANXA2 |

TABLE 4
Fold-change of mesenchymal stem cell markers.

| | Fold change | Marker |
|---|---|---|
| 1 | 3.768251304 | UBE2S |
| 2 | 2.776112783 | ASPM |
| 3 | 2.673339514 | PRC1 |
| 4 | 2.637550348 | TM4SF1 |
| 5 | 2.54365377 | UBE2C |
| 6 | 2.490291489 | FABP5 |
| 7 | 2.275032428 | TNFRSF12A |
| 8 | 2.254703228 | S100A16 |
| 9 | 2.207883103 | ODC1 |
| 10 | 2.203845375 | ENO1 |
| 11 | 2.200503383 | TMEM158 |
| 12 | 2.170667867 | HMGB2 |
| 13 | 2.117943707 | NME1 |
| 14 | 2.003429837 | DNMT1 |
| 15 | 1.940727884 | CEP55 |
| 16 | 1.876140375 | CKAP5 |
| 17 | 1.81220581 | AXL |
| 18 | 1.811180989 | DIAPH3 |
| 19 | 1.785021392 | PCNA |
| 20 | 1.75149531 | YBX1 |
| 21 | 1.698479327 | C1QBP |
| 22 | 1.673708897 | EIF5A |
| 23 | 1.662490454 | DNAJC9 |
| 24 | 1.604480097 | PSMD2 |
| 25 | 1.597632692 | ARHGAP18 |
| 26 | 1.591220561 | NES |
| 27 | 1.591033625 | UAP1 |
| 28 | 1.58136169 | ARPCIA |
| 29 | 1.575080439 | LY6K |
| 30 | 1.564745512 | EZR |
| 31 | 1.557190866 | ARHGAP22 |
| 32 | 1.53509994 | PDAP1 |
| 33 | 1.529631067 | WDR1 |
| 34 | 1.501692154 | EMP3 |
| 35 | 1.472935246 | CAVIN3 |
| 36 | 1.455780893 | PSMC5 |
| 37 | 1.455456084 | CCT3 |
| 38 | 1.454771986 | MSN |
| 39 | 1.445675108 | COMMD4 |
| 40 | 1.444319419 | ANXA2 |
| 41 | 1.437494896 | CDC42EP3 |
| 42 | 1.431775091 | PSMD9 |
| 43 | 1.431241115 | COPRS |
| 44 | 1.419217662 | PALM2-AKAP2 |

1. A method for identifying a multipotent mesenchymal stem cell subpopulation, among a population of cells, comprising the steps of:
i. providing the population of cells; and
ii. contacting and determining the population of cells with at least one probe, capable of identifying at least one marker, selected from a group consisting of: ubiquitin conjugating enzyme E2 S (UBE2S), assembly factor for spindle microtubules (ASPM), protein regulator of cytokinesis 1 (PRC1), ubiquitin conjugating enzyme E2 C (UBE2C), fatty acid binding protein 5 (FABP5), TNF receptor superfamily member 12A (TNFRSF12A), S100 calcium binding protein A16 (S100A16), ornithine decarboxylase 1 (ODC1), enolase 1 (ENO1), NME/NM23 nucleoside diphosphate kinase 1 (NME1), centrosomal protein 55 (CEP55), diaphanous related formin 3 (DIAPH3), Y-box binding protein 1 (YBX1), complement C1q binding protein (C1QBP), eukaryotic translation initiation factor 5A (EIF5A), DnaJ heat shock protein family (DNAJC9), Rho GTPase activating protein 18 (ARHGAP18), UDP-N-acetylglucosamine pyrophosphorylase 1 (UAP1), actin related protein 2/3 complex subunit 1A (ARPC1A), lymphocyte antigen 6 family member K (LY6K), ezrin (EZR), Rho GTPase activating protein 22 (ARHGAP22), PDGFA associated protein 1 (PDAP1), epithelial membrane protein 3 (EMP3), caveolae associated protein 3 (CAVIN3), proteasome 26S subunit, ATPase 5 (PSMC5), chaperonin containing TCP1 subunit 3 (CCT3), COMM domain containing 4 (COMMD4), annexin A2 (ANXA2), proteasome 26S subunit, non-ATPase 9 (PSMD9), coordinator of PRMT5 and differentiation stimulator (COPRS), PALM2 and AKAP2 fusion (PALM2-AKAP2), cytoskeleton associated protein 5 (CKAP5), CDC42 effector protein 3 (CDC42EP3), AXL receptor tyrosine kinase (AXL), moesin (MSN), WD repeat domain 1 (WDR1), DNA methyltransferase 1 (DNMT1), high mobility group box 2 (HMGB2), nestin (NES), proliferating cell nuclear antigen (PCNA), proteasome 26S subunit, non-ATPase 2 (PSMD2), Transmembrane Protein 158 (TMEM158), Transmembrane 4 L Six Family Member 1 (TM4SF1), and any combination thereof, thereby identifying a multipotent mesenchymal stem cell subpopulation, among a population of cells.

2. The method of claim 1, wherein said population of cells comprises gastrointestinal tract mucosa mesenchymal stromal cells, comprising said multipotent mesenchymal stem cell, and at least one of: a fibroblast and a myofibroblast cell.

3. The method of claim 2, wherein said gastrointestinal tract is the oral cavity.

4. The method of claim 2, wherein said mucosa is the lamina propria.

5. The method of claim 1, wherein said population of cells is provided by separation of the connective tissue comprising said lamina propria, from the epithelium of said mucosal tissue.

6. The method of claim 4, wherein said lamina propria is from a masticatory oral mucosa.

7. The method of claim 1, wherein said contacting is with a panel of probes, identifying a plurality of markers selected from a group consisting of: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2, TMEM158, and TM4SF1.

8. The method of claim 1, wherein said identifying comprises isolating, and optionally wherein said isolating is by cell sorting.

9. (canceled)

10. The method of claim 1, wherein said probe comprises one of: an antibody, an antibody fragment, a small molecule, and any combination thereof, conjugated to detectable tracer, comprising: a fluorophore, a chromophore, a magnetic bead, or any combination thereof.

11. The method of claim 8, for treating a disorder or a disease in a subject in need thereof, and optionally wherein said disorder or disease in a subject in need thereof, is selected from: a skin disorder, an autoimmune or an inflammatory disease, a nerve damage, an oral or craniofacial disorder, and a bone or a cartilage disorder.

12. (canceled)

13. A kit for identification of a multipotent mesenchymal stem cell subpopulation, among a population of cells, the kit comprising at least one probe, capable of identifying at least one marker, selected from a group consisting of: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2, TMEM158, TM4SF1, and any combination thereof.

14. The kit of claim 13, wherein said population of cells comprises gastrointestinal tract mucosa mesenchymal stromal cells, comprising said multipotent mesenchymal stem cell, and at least one of: a fibroblast and a myofibroblast cell, and optionally wherein: (i) said gastrointestinal tract is the oral cavity: (ii) said mucosa is the lamina propria, and optionally said lamina propria is from a masticatory oral mucosa; or both (i) and (ii).

15.-17. (canceled)

18. The kit of claim 13, wherein said at least one probe is a panel of probes, identifying a plurality of markers selected from a group consisting of: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2, TMEM158, and TM4SF1.

19. The kit of claim 13, wherein said identifying comprises isolation, and optionally wherein said isolation is by cell sorting.

20. (canceled)

21. The kit of claim 18, wherein said probe comprises one of: an antibody, an antibody fragment, a small molecule, and any combination thereof, conjugated to detectable tracer, comprising: a fluorophore, a chromophore, a magnetic bead, or any combination thereof.

22. A pharmaceutical composition comprising a mesenchymal stem cell subpopulation, and an acceptable carrier, wherein said mesenchymal stem cell subpopulation:
(a) constitutes at least 50% of cells within the pharmaceutical composition; and
(b) is characterized by expression of at least one marker, selected from a group consisting of: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, NT5E, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2, TMEM158, TM4SF1, and any combination thereof.

23. The pharmaceutical composition of claim 22, wherein any one of: (i) said mesenchymal stem cell subpopulation is derived from gastrointestinal tract mucosa mesenchymal stromal cells, comprising said multipotent mesenchymal stem cell, and at least one of: a fibroblast and a myofibroblast cell, and optionally wherein: said gastrointestinal tract is the oral cavity, said mucosa is the lamina propria, aid lamina propria is from a masticatory oral mucosa, or any combination thereof: (ii) said mesenchymal stem cell subpopulation comprises: a multipotent phenotype, an anti-inflammatory characteristic, an immunomodulatory characteristic, or any combination thereof, and optionally wherein said multipotent phenotype is an ability to differentiate into at least an osteogenic lineage: (iii) said expression is, independently, above a predetermined threshold, and optionally wherein said above a predetermined threshold expression, is at least 1.2-fold induction in the expression, compared to expression in a mesenchymal stromal non-stem cell, within said composition, comprising: a fibroblast, a myofibroblast, or any combination thereof; and (iv) any combination of (i) to (iii).

24.-30. (canceled)

31. The pharmaceutical composition of claim 22, characterized by above a predetermined threshold expression of a plurality of markers selected from a group consisting of: UBE2S, ASPM, PRC1, UBE2C, FABP5, TNFRSF12A, S100A16, ODC1, ENO1, NME1, CEP55, DIAPH3, YBX1, C1QBP, EIF5A, DNAJC9, ARHGAP18, UAP1, ARPC1A, LY6K, EZR, ARHGAP22, PDAP1, EMP3, CAVIN3, PSMC5, CCT3, COMMD4, ANXA2, PSMD9, COPRS, PALM2-AKAP2, CKAP5, CDC42EP3, AXL, MSN, WDR1, DNMT1, HMGB2, NES, PCNA, PSMD2, TMEM158, and TM4SF1.

32. A method for treating a subject suffering from a disorder or a disease, comprising administering to said subject a therapeutically effective amount of the pharmaceutical composition of claim 22, and optionally wherein said disorder or disease is selected from the group consisting of: a skin disorder, an autoimmune or an inflammatory disease, nerve damage, an oral or craniofacial disorder, and a bone or a cartilage disorder.

33. (canceled)
